(19) United States
(12) Patent Application Publication
(10) Pub. No.: US 2024/0173456 A1
(43) Pub. Date: May 30, 2024

(54) THERAPEUTIC COMPOSITIONS FOR WOUND TREATMENT

(71) Applicant: GlycoSurf, Inc., Salt Lake City, UT (US)

(72) Inventors: Chett Boxley, Park City, UT (US); Brenda Padilla, Casa Grande, AZ (US); Taylor Reese Gilmore, Flagstaff, AZ (US); Robert Shawn Kellar, Flagstaff, AZ (US)

(21) Appl. No.: 18/494,584

(22) Filed: Oct. 25, 2023

Related U.S. Application Data
(60) Provisional application No. 63/419,305, filed on Oct. 25, 2022.

(51) Int. Cl. A61L 15/44; A61L 15/22

(52) U.S. Cl. CPC A61L 15/44; A61L 15/225; A61L 2300/22

(57) ABSTRACT

Described herein are therapeutic compositions comprising a rhamnolipid impregnated within an electrospun support, and methods of using the same. Exemplary therapeutic compositions may be used for wound treatment.

### CROSS-REFERENCE TO RELATED APPLICATION(S)

This application claims priority to U.S. Provisional Patent Application No. 63/419,305, filed on Oct. 25, 2022, the entire contents of which are fully incorporated herein by reference.

### TECHNICAL FIELD

The present disclosure relates to therapeutic compositions comprising a rhamnolipid impregnated within an electrospun support, and methods of using the same. Exemplary therapeutic compositions may be used for wound treatment.

### INTRODUCTION

Exposure to metal ions, such as depleted uranium (DU), has been shown to hinder the natural wound healing process through a variety of cytotoxic mechanisms, e.g., decreased cell locomotion, metabolic, and apoptotic death, in fibroblast cells. Current literature is silent on therapeutic solutions to counteract these negative effects. What is needed are improved compositions and methods for treating wounds contaminated with metal ions, e.g., wound contaminated with depleted uranium (DU).

### SUMMARY

In some aspects, the present disclosure provides a therapeutic composition. Exemplary therapeutic compositions may comprise a rhamnolipid, and an electrospun support comprising a biomaterial, wherein the rhamnolipid is impregnated within the electrospun support. The electrospun support may comprise a naturally occurring biomaterial, such as a naturally occurring biomaterial comprising collagen, elastin, albumin, casein, fibrin, fibroin, keratin, tropoelastin, or a combination thereof. The electrospun support may comprise a synthetic biomaterial, such as a synthetic biomaterial comprising polylactide (PLA), polyglycolide (PGA), poly(lactide-co-glycolide) (PLGA), polycaprolactone (PCL), polyvinylidene fluoride (PVDF), polydioxanone trimethylene carbonate (PTMC), poly(Bisphenol A carbonate) (PC), polycyanoacrylate, a polyhybroxyalkonate (PHA), a poly(ortho ester) (POE), or a combination thereof. The rhamnolipid may comprise a mono-rhamnolipid, a di-rhamnolipid, or a combination thereof. The rhamnolipid may comprise a rhamnolipid of formula (I):

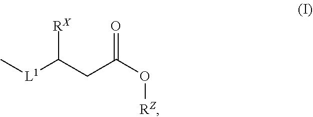

wherein:
R^{X} is

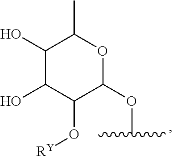

where R^{Y} is hydrogen or

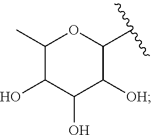 [SMILES: *C1OC(C)C(O)C(O)C1O]

R^{Z} is hydrogen or

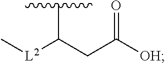

L¹ is C₂₋₁₈alkylene or C₂₋₁₈alkenylene; and
L² is C₂₋₁₈alkylene or C₂₋₁₈alkenylene.

### R^{X} may be

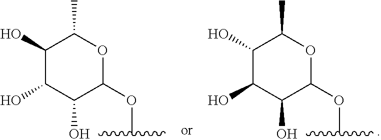 [SMILES: *OC1O[C@@H](C)[C@H](O)[C@@H](O)[C@H]1O ; *OC1O[C@H](C)[C@@H](O)[C@H](O)[C@@H]1O]

### R^{X} may be

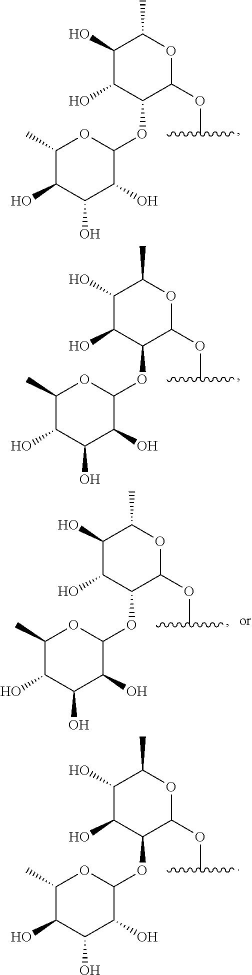

The rhamnolipid of formula (I) may be a rhamnolipid of formula (I-a), (I-b), (I-aa), (I-bb), (I-ab), or (I-ba):

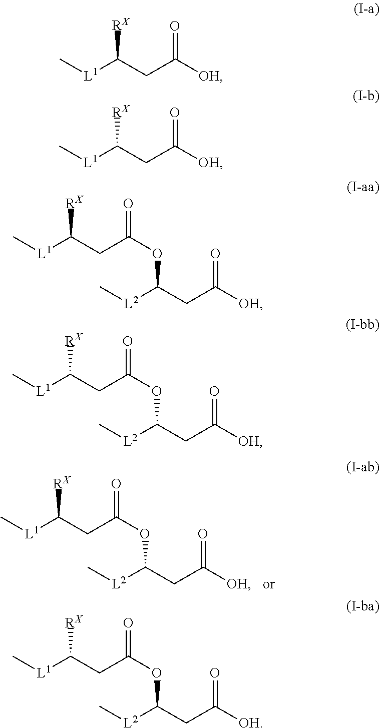

L¹ may be C₂₋₁₈alkylene or C₂₋₁₈alkenylene. L² may be C₂₋₁₈alkylene or C₂₋₁₈alkenylene.

The rhamnolipid of formula (I) may have enantiomeric purity in excess of 90.0%.

The rhamnolipid may comprise a synthetically prepared rhamnolipid. The rhamnolipid may comprise a naturally occurring rhamnolipid. The rhamnolipid may be present in the therapeutic composition at a concentration of greater than 1 μM. The rhamnolipid may be present in the therapeutic composition at a concentration of less than 10,000 μM. The rhamnolipid may be present in the therapeutic composition at a concentration of 3 μM to 100 μM. The therapeutic composition may have a pH of less than 8, such as a pH of 4.5-6.5. The therapeutic composition may further comprise an antibiotic, a steroid, an analgesic, a cannabinoid, a growth factor, or a combination thereof.

In other aspects, the present disclosure provides a wound care dressing. The wound care dressing may comprise the therapeutic composition and a wound dressing material. The wound dressing material may be a bandage, a wipe, a sponge, a mesh, a gauze, a patch, a pad, a tape, or a wrap.

In other aspects, the present disclosure provides a method of treating a wound of a subject. The method of treating a wound of a subject may comprise applying the therapeutic composition to a surface of the subject's wound. The wound may contain one or more metal ions. The wound may contain depleted uranium.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 schematically illustrates the cellular migration assay process, where fibroblast cells are grown inside a biosafety cabinet (A), in a flask (B), prior to subculturing into working 12-well plates (C), and then the monolayer of neonatal human dermal fibroblast (hDFn) cells (D) is “scratched” using a pipette tip (E), thus creating the mock wound (F).

FIG. 2 depicts the general process of electrospinning, where a polymer protein solution is charged with a high voltage from a positively charged needle onto a negatively charged aluminum collector where nanofibers are encouraged to form.

FIG. 3A is a bar graph showing percent closure rates of hDFn cells that were exposed to 0.1 μM depleted uranium (DU), then treated with rhamnolipids (6.25 and 50 μM) 24 hours before a mock wound was created using the bench top cell migration assay. The bar graph illustrates average cell migration 12 hours post-wound+/−standard error of the mean (n=3 for each treatment group, *=p<0.05).

FIG. 3B is a bar graph showing percent closure rates of hDFn cells that were exposed to 1.0 μM depleted uranium (DU), then treated with rhamnolipids (6.25 and 50 μM) 24 hours before a mock wound was created using the bench top cell migration assay. The bar graph illustrates average cell migration 12 hours post-wound+/−standard error of the mean (n=3 for each treatment group, *=p<0.05).

FIG. 3C is a bar graph showing percent closure rates of hDFn cells that were exposed to 10 μM depleted uranium (DU), then treated with rhamnolipids (6.25 and 50 μM) 24 hours before a mock wound was created using the bench top cell migration assay. The bar graph illustrates average cell migration 12 hours post-wound+/−standard error of the mean (n=3 for each treatment group, *=p<0.05).

FIG. 4 is a bar graph showing percent closure rates of hDFn cells with and without rhamnolipid treatment. Cells were treated with rhamnolipids (6.25 and 50 μM) 24 hours before the creation of a mock wound. The bar graph illustrates average cell migration 12 hours post-wound+/−standard error of the mean (n=3 for each treatment group, *=p<0.05).

FIGS. 5A-5E are scanning electron microscopy (SEM) images of cell-free electrospun scaffolds.

FIG. 5A is a SEM image of cell-free electrospun scaffold without rhamnolipid (control).

FIG. 5B is an SEM image of a cell-free electrospun scaffold impregnated with 1% by weight (wt %) rhamnolipids.

FIG. 5C is an SEM image of a cell-free electrospun scaffold impregnated with 2.5 wt % rhamnolipids.

FIG. 5D is an SEM image of a cell-free electrospun scaffold impregnated with 5 wt % rhamnolipids.

FIG. 5E is an SEM image of a cell-free electrospun scaffold impregnated with 10 wt % rhamnolipids.

FIGS. 6A-6E are SEM images of electrospun scaffolds cellularized with HDFn cells. HDFn cells are highlighted in blue.

FIG. 6A is a SEM image of a cellularized electrospun scaffold without any rhamnolipid.

FIG. 6B is a SEM image of a cellularized electrospun scaffold impregnated with 1 wt % rhamnolipids.

FIG. 6C is a SEM image of a cellularized electrospun scaffold impregnated with 2.5 wt % rhamnolipids.

FIG. 6D is a SEM image of a cellularized electrospun scaffold impregnated with 5 wt % rhamnolipids.

FIG. 6E is a SEM image of a cellularized electrospun scaffold impregnated with 10 wt % rhamnolipids.

FIG. 7A is a bar graph showing metabolic activity analysis for hDFn cells with and without rhamnolipids. The bar graphs show the average relative fluorescence unit (RFU) values+/−standard error of the mean (n=6 for each treatment group, *=p<0.05).

FIG. 7B is a bar graph showing DNA content of hDFn cells with and without rhamnolipids. The bar graphs show the average RFU values+/−standard error of the mean (n=6 for each treatment group, *=p<0.05).

FIG. 8A is a bar graph showing metabolic activity analysis for hDFn cells with and without varying concentrations of rhamnolipids. The bar graph shows the average RFU values+/−standard error of the mean (n=12 for each treatment group, *=p<0.05).

FIG. 8B is a bar graph showing and DNA content analysis for hDFn cells with and without varying concentrations of rhamnolipids. The bar graph shows the average RFU values+/−standard error of the mean (n=12 for each treatment group, *=p<0.05).

FIG. 9 shows SEM images of scratch assay results comparing the wound healing capabilities of rhamnolipid compositions versus thioglycolipid compositions over a 24-hour time period.

FIG. 10A is a bar graph showing percent closure rates of rhamnolipid treated cells used in a scratch assay over 24 hours. Measurements were taken every four hours. Rhamnolipid concentrations of 6.25 μM, 12.5 μM, 25 μM and 50 μM were evaluated.

FIG. 10B is a bar graph showing percent closure rates of thioglycolipid treated cells used in a scratch assay over 24 hours. Measurements were taken every four hours. Thioglycolipid concentrations of 6.25 μM, 12.5 μM, 25 μM and 50 μM were evaluated.

### DETAILED DESCRIPTIONS

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various way.

### I. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkylene,” as used herein, refers to a divalent group derived from a straight or branched saturated chain hydrocarbon, for example, of 1 to 6 carbon atoms. Representative examples of alkylene include, but are not limited to, —CH₂—, —CD₂—, —CH₂CH₂—, —C(CH₃)(H)—, —C(CH₃)(D)—, —CH₂CH₂CH₂—, —CH(CH₃)CH₂CH₂—, —C(CH₃)₂CH₂CH₂—, —CH₂C(CH₃)₂CH₂—, —CH₂CH₂CH₂CH₂—, and —CH₂CH₂CH₂CH₂CH₂—.

The term “alkenylenyl,” as used herein, refers to a divalent alkenyl group, examples of which include but are not limited to —CH═CH—, —CH═CH—CH₂—, —CH═CH—CH₂—CH₂—and —CH₂—CH═CH—CH₂—. An alkenylenyl group may be optionally substituted with one or more substituents.

The term “racemic mixture” or “racemate” means a mixture of equal quantities of two enantiomers or substances that have dissymmetric molecular structures that are mirror images of one another.

The term “enantiomeric purity” (i.e., optical purity), as used herein, refers to the fractional excess of one enantiomer over the other.

### II. Therapeutic Compositions

Described herein are therapeutic compositions comprising a rhamnolipid and an electrospun support, wherein the rhamnolipid is impregnated within the electrospun support. The disclosed therapeutic compositions may be suitable for administration to a subject (such as a patient, which may be a human or non-human animal, such as a mammal). The therapeutic compositions may include a “therapeutically effective amount” of the rhamnolipid, wherein the rhamnolipid is impregnated in an electrospun support. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects are outweighed by the therapeutically beneficial effects.

### A. Rhamnolipids

Exemplary therapeutic compositions comprise a rhamnolipid. Rhamnolipids generally comprise one or more rhamnose moieties and an ester-linked di-lipid tail. Exemplary therapeutic compositions may include a rhamnolipid comprising a mono-rhamnolipid, a di-rhamnolipid, or a combination thereof. The particular rhamnolipid(s) included in the therapeutic composition may depend on the specific wound being treated.

In various instances, the rhamnolipid may comprise, without limitation, a rhamnolipid of formula (I):

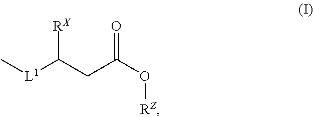

wherein:
R^{X} is

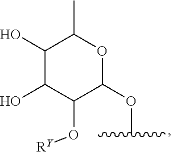

where R^{Y} is hydrogen or

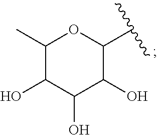 [SMILES: *C1OC(C)C(O)C(O)C1O]

R^{Z} is hydrogen or

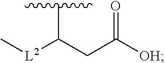

L¹ is C₂₋₁₈alkylene or C₂₋₁₈alkenylene; and
L² is C₂₋₁₈alkylene or C₂₋₁₈alkenylene.

In various instances, R^{X} may be:

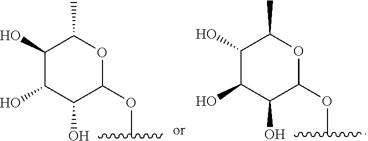 [SMILES: *OC1O[C@@H](C)[C@H](O)[C@@H](O)[C@H]1O ; *OC1O[C@H](C)[C@@H](O)[C@H](O)[C@@H]1O]

In other instances, R^{X} may be:

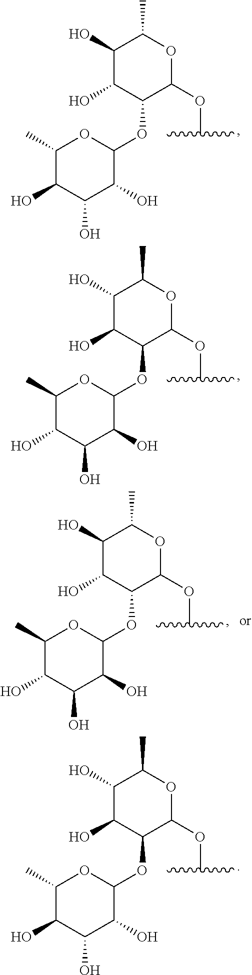

In some instances, the rhamnolipid of formula (I) may be a rhamnolipid of formula (I-a), (I-b), (I-aa), (I-bb), (I-ab), or (I-ba):

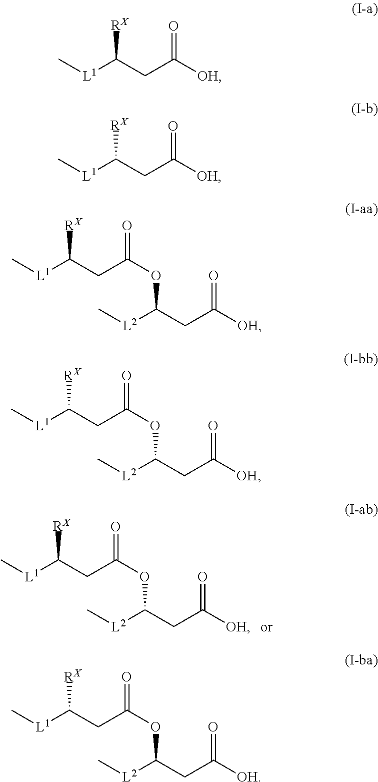

In some instances, L¹ may be C₂₋₁₈alkylene or C₂₋₁₈alkenylene and L² may be C₂₋₁₈alkylene or C₂₋₁₈alkenylene.

In some instances, the rhamnolipid may be present as a racemic mixture. In other instances, the rhamnolipid may have enantiomeric purity in excess of 55%; in excess of 60%; in excess of 65%; in excess of 70%; in excess of 75%; in excess of 80%; or in excess of 85%. In some instances, the rhamnolipid of formula (I) may have enantiomeric purity in excess of 90.0%.

In various instances, the rhamnolipid may comprise a synthetically prepared rhamnolipid. Exemplary synthetically prepared rhamnolipids may be prepared as described in U.S. Pat. Nos. 9,499,575 and 11,117,914, which are both incorporated herein by reference in their entirety.

In some instances, the rhamnolipid many comprise a naturally occurring rhamnolipid. Exemplary naturally occurring rhamnolipids include rhamnolipids found in the Gram-negative bacteria such as Acinetobacter calcoaceticus, Enterobacter asburiae, Enterobacter hormaechei, Pantoea stewartii, and Pseudomonas aeruginosa. Exemplary rhamnolipids may also include rhamnolipids biosynthetically produced via the mutant strain of bacteria, P. aeruginosa ATCC 9027 (a mutant that produces specifically monorhamnolipids) produces about 40 different monorhamnolipid congeners with a variety of saturated and unsaturated lipid chain lengths (ranging from C₆ to C₁₈).

In various instances, the rhamnolipid may be present in the therapeutic composition at a concentration of greater than 1 μM. In various instances, the rhamnolipid may be present in the therapeutic composition at a concentration of less than 10,000 μM. In various instances, the rhamnolipid may be present in the therapeutic composition at a concentration of 3 μM to 100 μM. In some instances, the rhamnolipid may be present in the therapeutic composition at a concentration of 5 μM to 95 μM; 10 μM to 90 μM; 15 μM to 85 μM; 20 μM to 80 μM; 25 μM to 75 μM; 30 μM to 70 μM; 35 μM to 65 μM; 40 μM to 60 μM; or 45 μM to 55 μM. In some instances, the rhamnolipid may be present in the therapeutic composition at a concentration of no greater than 100 μM; no greater than 90 μM; no greater than 80 μM; no greater than 70 μM; no greater than 60 μM; no greater than 50 μM; no greater than 40 μM; no greater than 30 μM; no greater than 20 μM; no greater than 10 μM; or no greater than 5 μM. In some instances, the rhamnolipid may be present in the therapeutic composition at a concentration of no less than 3 μM; no less than 5 μM; no less than 10 μM; no less than 20 μM; no less than 30 μM; no less than 40 μM; no less than 50 μM; no less than 60 μM; no less than 70 μM; no less than 80 μM; or no less than 90 04.

In exemplary therapeutic compositions, the rhamnolipid is incorporated, e.g., impregnated, within an electrospun support.

### B. Electrospun Supports

The term “electrospun support,” as used herein, means a construct, device, scaffold, membrane, sheet, or tube that is electrospun and acts as a foundation or structure for the rhamnolipids described herein. The terms “electrospinning” or “electrospun,” as used herein refers to any method where materials are streamed, sprayed, sputtered, dripped, or otherwise transported in the presence of an electric field. The electrospun material can be deposited from the direction of a charged container towards a grounded target, or from a grounded container in the direction of a charged target. In particular, the term “electrospinning” means a process in which fibers are formed from a charged solution comprising at least one natural biological material, at least one synthetic polymer material, or a combination thereof by streaming the electrically charged solution through an opening or orifice towards a grounded target.

Exemplary therapeutic compositions may comprise an electrospun support comprising one or more biomaterials. The term “biomaterial,” as used herein, means a material that materials that has been designed to interface with biological systems, for the treatment, augmentation, or replacement of biological functions. The specific biomaterials used for the electrospun support may be adjusted in accordance with the needs and specifications of the rhamnolipids and/or additional therapeutic agents incorporated therein. Furthermore, the specific electrospun support included in the therapeutic composition may depend on the specific wound being treated.

In various instances, the electrospun support may comprise one or more naturally occurring biomaterials. Suitable naturally occurring biomaterials may include, without limitation, amino acids, polypeptides, denatured peptides such as gelatin from denatured collagen, carbohydrates, lipids, nucleic acids, glycoproteins, lipoproteins, glycolipids, glycosaminoglycans, and proteoglycans. In various instances, the biomaterial may comprise one or more extracellular matrix proteins. Exemplary extracellular matrix proteins may include, without limitation, collagen, elastin, agarose, albumin, alginate, casein, elastin, fibrin, fibroin, fibronectin, gelatin, keratin, laminin, pectin, tropoelastin, cellulose, chitosan, chitin, and combinations thereof

In various instances, the electrospun support may comprise one or more synthetic biomaterials. Suitable synthetic biomaterials include, but are not limited to, synthetic polymers such as polylactide (PLA), polyglycolide (PGA), poly(lactide-co-glycolide) (PLGA), polycaprolactone (PCL), polyvinylidene fluoride (PVDF), polydioxanone trimethylene carbonate (PTMC), a polyhybroxyalkonates (PHA) (e.g., polyhydroxybutyrate (PHB), poly(γ-ethyl-L-glutamate), poly(Bisphenol A carbonate) (PC), poly(ortho esters) (POEs), polycyanoacrylate, and combinations thereof In various instances, the electrospun support may comprise a co-polymer. The term “co-polymer” as used herein is intended to encompass co-polymers, ter-polymers, and higher order multiple polymer compositions formed by block, graph, or random combination of polymeric components.

### C. Additional Therapeutic Agents

In various instances, exemplary therapeutic compositions may further include an additional therapeutic agent. Exemplary additional therapeutic agents may include, without limitation, an antibiotic, a steroid, an analgesic, a cannabinoid, a growth factor, and combinations thereof. When present, the specific additional therapeutic agent(s) included in the therapeutic composition may depend on the specific wound being treated.

Exemplary antibiotics may include, without limitation, Amikacin disulfate salt, Amikacin hydrate, Anisomycin from Streptomyces griseolus, Apramycin sulfate salt, Azithromycin, Blasticidine S hydrochloride, Brefeldin A, Brefeldin A from Penicillium brefeldianum, Butirosin sulfate salt, Butirosin. A from Bacillus vitellinus, Chloramphenicol, Chloramphenicol base, Chloramphenicol succinate sodium salt, Chlortetracycline hydrochloride, Chlortetracycline hydrochloride from Streptomyces aureofaciens, Clidamycin 2-phosphate, Clindamycin hydrochloride, Clotrimazole, Cycloheximide from microbial, Demeclocycline hydrochloride, Dibekacin sulfate salt, Dihydrostreptomycin sesquisulfate, Dihydrostreptomycin solution, Doxycycline hyclate, Duramycin from Streptoverticillium cinnamoneus, Emetine dihydrochloride hydrate), Erythromycin, Erythromycin USP, Erythromycin powder, Erythromycin, Temephos, Erythromycin estolate, Erythromycin ethyl succinate, Erythromycin standard solution, Erythromycin stearate, Fusidic acid sodium salt, G 418 disulfate salt, G 418 disulfate salt powder, G 418 disulfate salt solution liquid, Gentamicin solution liquid, Gentamicin solution, Gentamicin sulfate Micromonospora purpurea, Gentamicin sulfate salt, Gentamicin sulfate salt powder USP, Gentamicin-Glutamine solution liquid, Helvolic acid from Cephalosporium caerulens, Hygromycin B Streptomyces hygroscopicus, Hygromycin B Streptomyces hygroscopicus powder, Hygromycin B solution Stremomyces hygroscopicus, Josamycin, Josamycin solution, Kanamycin B sulfate salt, Kanamycin disulfate salt from Streptomyces kanamycencus, Kanamycin monosulfate from Streptomyces kanamyceticus, Kanamycin monosulfate from Streptomyces kanamyceticus powder USP, Kanamycin solution from Streptomyces kanamyceticus, Kirromycin from Streptomyces collinus, Lincomycin hydrochloride, Lincomycin standard solution, Meclocycline sulfosalicylate salt, Mepartricin, Midecamycin from Streptomyces mycarofaciens, Minocydine hydrochloride crystalline, Neomycin solution, Neomycin trisulfate salt hydrate, Neomycin trisulfate salt hydrate powder, Neomycin trisulfate salt hydrate USP powder, Netilmicin sulfate salt, Nitrofurantoin crystalline, Nourseothricin sulfate, Oleandomycin phosphate salt, Oleandomycin triacetate, Oxytetracycline dihydrate, Oxytetracycline hemicalciuin salt; Oxytetracycline hydrochloride, Paromomycin sulfate salt, Puromycin dihydrochloride from Streptomyces alboniger, Rapamycin from Streptomyces hygroscopicus, Ribostamycin sulfate salt, Rifampicin, Rifamycin SV sodium salt, Rosamicin Micromonospora rosaria, Sisomicin sulfate salt, Spectinomycin dihydrochloride hydrate, Spectinomycin dihydrochloride hydrate powder, Spectinomycin dihydrochloride pentahydrate, Spiramycin, Spiramycin from Streptomyces sp., Spiramycin solution, Streptomycin solution, Streptomycin sulfate salt, Streptomycin sulfate salt powder, Tetracycline, Tetracycline hydrochloride, Tetracycline hydrochloride USP, Tetracycline hydrochloride powder, Thiamphenicol, Thiostrepton from Streptomyces azureus, Tobramycin, Tobramycin sulfate salt, Tunicamycin A1 homolog, Tunicamycin C2 homolog, Tunicamycin Streptomyces sp., Tylosin solution, Tylosin tartrate, Viomycin sulfate salt, Virginiamycin M1, (S)-(+)-Camptothecin, 10-Deacetylbaccatin III from Taxus baccata, 5-Azacytidine, 7-Aminoactinomycin D, 8-Quinolinol crystalline, 8-Quinolinol hemisulfate salt crystalline, 9-Dihydro-13-acetylbaccatin III from Taxus canadensis, Aclarubicin, Aclarubicin hydrochloride, Actinomycin D from Streptomyces sp., Actinomycin I from Streptomyces antibioticus, Actinomycin V from Streptomyces antibioticus, Aphidicolin Nigrospora sphaerica, Bafilomycin A1 from Streptomyces griseus, Bleomycin sulfate from Streptomyces verticillus, Capreomycin sulfate from Streptomyces capreolus, Chromomycin A3 Streptomyces griseus, Cinoxacin, Ciprofloxacin BioChemika, cis-Diammineplatinum(II) dichloride, Coumermycin A1, Cytochalasin B Helminthosporium dematioideum, Cytochalasin D Zygosporium mansonii, Dacarbazine, Daunorubicin hydrochloride, Daunorubicin hydrochloride USP, Distamycin A hydrochloride from Streptomyces distallicus, Doxorubicin hydrochloride, Echinomycin, Echinomycin BioChemika, Enrofloxacin BioChemika, Etoposide, Etoposide solid, Flumequine Formycin, Fumagillin from Aspergillus fumigatus, Ganciclovir, Gliotoxin from Gliocladium fimbriatum, Lomefloxacin hydrochloride, Metronidazole purum, Mithramycin A from Streptomyces plicatus, Mitomycin C Streptomyces caespitosus, Nalidixic acid, Nalidixic acid sodium salt. Nalidixic acid sodium salt powder, Netropsin dihydrochloride hydrate, Nitrofurantoin, Nogalamycin from Streptomyces nogalater, Nonactin from Streptomyces tsusimaensis, Novobiocin sodium salt, Ofloxacin, Oxolinic acid, Paclitaxel from Taxus yannanensis, Paclitaxel from Taxus brevifolia, Phenazine methosulfate, Phileomycin Streptomyces verticullus, Pipemidic acid, Rebeccamycin from Saccharothrix acrocolonigenes, Sinefungin Streptonigrin from Streptomyces flocculus, Streptozocin, Succinylsulfathiazole, Sulfadiazine, Sulfadimethoxine, Sultaguanidine purum, Sulfamethazine, Sulfamonomethoxine, Sulfanilamide, Sulfaquinoxaline sodium salt, Sulfasalazine, Sulfathiazole sodium salt, Trimethoprim, Trimethoprim lactate salt, Tubercidin from Streptomyces tubercidicus, 5-Azacytidine, Cordycepin, Formycin A, (+)-6-Aminopenicillanic acid, 7-Aminodesacetoxycephalosporanic acid, Amoxicillin, Ampicillin, Ampicillin sodium salt, Ampicillin trihydrate, Ampicillin trihydrate USP, Aziocillin sodium salt, Bacitracin Bacillus licheniformis, Bacitracin zinc salt Bacillus lichenitbrinis, Carbenicillin disodium salt, Cefaclor, Cefamandole lithium salt, Cefamandole notate, Cefamandole sodium salt, Cefazolin sodium salt, Cefinetazole sodium salt, Cefoperazone sodium salt, Cefotaxime sodium salt, Cefsulodin sodium salt, Cefsulodin sodium salt hydrate, Ceftriaxone sodium salt, Cephalexin hydrate, Cephalosporin C zinc salt, tephalothin sodium salt, Cephapirin sodium salt Cephradine, Cloxacillin sodium salt, Cloxacillin sodium salt monohydrate, D-Penicillamine hydrochloride, DT Cycloserine microbial, D-Cycloserine powder, Dicloxacillin sodium salt monohydrate, D-Penicillamine, Econazole nitrate salt, Ethambutol dihydrochloride, Lysostaphin from Staphylococcus staphytolyticus, Moxalactam sodium salt, Nafcillin, sodium salt monohydrate, Nikkomycin, Z Streptomyces tendae, Nitrofurantoin crystalline, Oxacillin sodium salt, Penicillic acid powder, Penicillin G potassium salt, Penicillin G potassium salt powder, Penicillin G potassium salt, Penicillin C sodium salt hydrate powder, G sodium salt powder, Penicillin G sodium salt, Phenethicillin potassium salt, Phenoxymethyipenicillinic acid potassium salt, Phosphomycin sodium salt, Pipemidic acid, Piperacillin sodium salt, Ristomycin monosulfate, Vancomycin hydrochloride from Streptomyces orientalis, 2-Mercaptopyridine N-oxide sodium salt, 4-Bromocalcimycin A23187 BioChemika, Alamethicin Trichoderma viride, Amphotericin B Streptomyces sp., Amphotericin B preparation, Calcimycin A23187, Calcimycin A23187 hemi(calcium-magnesium) salt, Calcimycin A23187 hemicalcium salt, Calcimycin A23187 hemimagnesium salt, Chlorhexidine diacetate salt monohydrate, Chlorhexidine diacetate salt hydrate, Chlorhexidine digluconate, Clotrimazole, Colistin sodium methanesulfonate, Colistin sodium methanesulfonate from Bacillus colistinus, Colistin sulfate salt, Econazole nitrate salt, hydrocortisone 21-acetate, Filipin complex Streptomyces filipinensis, Gliotoxin from Gliocladium fimbriatum, Gramicidin A from Bacillus brevis, Gramicidin C from Bacillus brevis, Gramicidin from Bacillus aneurinolyticus (Bacillus brevis), ionomycin calcium salt Streptomyces conglobatus, Lasalocid A sodium salt, Lonomycin A sodium salt from Streptomyces ribosidificus, Monensin sodium salt, N-(6-Aminohexyl)-5-chloro-1-naphthalenesulfonamide hydrochloride, Narasin from Streptomyces auriofaciens, Nigericin sodium salt from Streptomyces hygroscopicus, Nisin from Streptococcus lactis, Nonactin from Streptomyces sp., Nystatin, Nystatin powder, Phenazine methosulfate, Pimaricin, Pimaricin from Streptomyces chattanoogensis, Polymyxin B solution, Polymyxin B sulfate salt, DL-Penicillamine acetone adduct hydrochloride monohydrate, Polymyxin B sulfate salt powder USP, Praziquantel, Salinomycin from Streptoinyces albus, Salinomycin from Streptomyces albus, Surfactin from Bacillus subtilis, Valinomycin, (+)-Usnic acid from Usnea dasypoga, (+)-Miconazole nitrate salt, (S)-(+)-Camptothecin, 1-Deoxymannojirimycin hydrochloride, 1-Deoxynojirimycin hydrochloride, 2-Heptyl-4-hydroxyquinoline N-oxide, Cordycepin, 1,10-Phenanthrofine hydrochloride monohydrate puriss, 6-Diazo-5-oxo-L-norleucine, 8-Quinolinol crystalline, 8-Quinolinol hemisulfate salt, Antimycin A from Streptomyces sp., Antimycin A1, Antimycin A2, Antimycin A3, Antipain, Ascomycin, Azaserine, Bafilomycin A1 from Streptomyces griseus, Bafilomycin B1 from Streptomyces species, Cerulenin BioChemika, Chloroquine diphosphate salt, Cinoxacin, Ciprofloxacin, Mevastatin BioChemika, Concanamycin A, Concanamycin A1, Streptomyces sp, Concanamycin C from Streptomyces species, Coumermycin A1, Cyclosporin A from Tolypocladium inflatum, Cyclosporin A, Econazole nitrate salt, Enrofloxacin, Etoposide, Flumequine, Formycin A, Furazolidone, Fusaric acid from Gibberella fujikuroi, Geldanamycin from Streptomyces hygroscopicus, Gliotoxin from Gliociadium fimbriatum, Gramicidin A from Bacillus brevis, Gramicidin C from Bacillus brevis, Gramicidin from Bacillus aneurinolyticus (Bacillus brevis), Gramicidin from Bacillus brevis, Herbimycin A from Streptomyces hygroscopicus, Indomethacin, Irgasan, Lomefloxacin hydrochloride, Mycophenolic acid powder, Myxothiazol BioChemika, N-(6-Aminohexyl)-5-chloro-1-naphthalenesulfonamide hydrochloride, Nalidixic acid, Netropsin dihydrochloride hydrate, Niclosamide, Nikkomycin BioChemika, Nikkomycin Z Streptomyces tendae, N-Methyl-1-deoxynojirimycin, Nogalamycin from Streptomyces nogalater, Nonactin quadrature. 80% from Streptomyces tsusimaensis, Nonactin from Streptomyces sp. Novobiocin sodium salt, Ofloxacin, Oleandomycin triacetate, Oligomycin Streptomyces diastatochromogenes, Oligomycin A, Oligomycin B, Oligomycin C, Oligomycin Streptomyces diastatochromogenes, Oxolinic acid, Pieriridin A from Streptomyces mobaraensis, Pipemidic acid, Radicicol from Diheterospora chlamydosporia solid, Rapamycin from Streptomyces hygroscopicus,Rebeccamycin from Saccharothrix aerocolonigenes, Sinefungin, Staurosporine Streptomyces sp., Stigmatellin, Succinylsulfathiazole, Sulfadiazine, Sulfadimethoxine, Sulfaguanidine purum, Sulfamethazine, Sulfamonomethoxine, Sulfanilamide, Sulfaquinoxaline sodium salt, Sulfasalazine, Sulfathiazole sodium salt, Triacsin C from Streptomyces sp., Trimethoprim, Trimethoprim lactate salt, Vineomycin A1 from Streptomyces albogriseolus subsp., Tectorigenin, and Paracelsin Trichoderma reesei.

Exemplary steroids may include, without limitation, corticosteroids, progestrogens, androgens, estrogens, neurosteroids, aminosteroids, or secosteroids.

Exemplary analgesics may include, without limitation, any analgesic compound suitable in a wound care composition, including, for example, a paracetamol, an MAID, an opioid, or an analgesic adjuvant, or any other suitable analgesic, including combinations of analgesics.

Exemplary cannabinoids may include any compound within the class that acts upon cannabinoid receptors (such as on the endocannahinoid system) in cells. A cannabinoid can include a synthetic cannabinoid, a phytocannabinoid, or an endocannabininoid. In some embodiments, a cannabinoid includes cannabidiols (including cannabidiol, cannabidiolic acid, cannabidiol monomethylether, cannabidiorcol, cannabidivarin, or cannabidivarinic acid), tetrahydrocannabinols (including delta-9-tetrahydrocannabinol, delta-9-cis-tetrahydrocannabinol, delta-9-tetrahydrocannabinol-C4, delta-9-tetrahydrocannabinolic acid A, delta-9-tetrahydrocannabinolic acid B, delta-9-tetrahydrocannabinolic acid C4, delta-9-tetrahydrocannabiorcol, delta-9-tetrahydrocannabiocolic acid, delta-9-tetrahydrocannabivarin, delta-9-tetrahydrocannabivarinic acid, tryhydroxy-delta-9-tetrahydrocannabinol, delta-8-tetrahydrocannabinol, or delta-8-tetrahydrocannabinolic acid), cannabichromenes (including cannabichromene, cannabichromenenic acid, cannabichromevarin, cannabichromenvarinic acid), cannabigerols (including cannabigerol, cannabigerol monomethylether, cannabigerolic acid, cannabigerolic acid monomethylether, cannabinerolic acid, cannabigerovarin, or cannabigerovarinic acid), cannabielsoins (including cannahielsoin, cannabielsoic acid A, or cannabielsoic acid B), cannabinols or cannabinodiols (including cannabinodiol, cannabinodivarin, cannabinol, cannabinol methylether, cannabinol-C2, cannabinol-C4, cannabinolic acid, cannabiorcool, or cannabivarin), cannabiniols (including cannabitriol, cannabitriolvarin, 10-ethoxy-9-hydroxy-delta-6a-tetrahydrocannabinol, or 8,9-dihydroxy-delta-6a-tetrayhydrocannabinol), cannabicyclols, 10-oxo-delta-6a-tetrahydrocannabinol, cannabichromanon, cannabifuran, cannabiglendol, cannabiripsol, cannbicitran, dehydrocannabifuran, cannabicitran, or 3,4,5,6-tetrahydro-7-hydroxy-alpha-alpha-trimethyl-9-n-propyl-2, 6-methano-2H-1-benzoxicin-5-methanol, or any analogue or derivative thereof.

Exemplary growth factors may include any growth factor, such as, but not limited to, epidermal growth factor (EGF), platelet derived growth factor (PDGF), fibroblast growth factor (FGF), transforming growth factors (TGF-α and TGF-β), nerve growth factor (NGF), erythropoietin (EPO), insulin-like growth factors (IGF-I and IGF-II), interleukin cytokines (IL-1α, IL-1β, IL-2, IL-3, IL-4, IL-5, IL-6, IL-7, IL-8, IL-9, IL-10, IL-11, IL-12, IL-13), interferons (IFN-α, IFN-β, and IFN-γ), tumor necrosis factors (INFα and INF-β), colony stimulating factors (GM-CSF and M-CSF).

### D. Pharmaceutically Acceptable Carriers

In some instances, exemplary therapeutic compositions may further comprise one or more pharmaceutically acceptable carriers. The term “pharmaceutically acceptable carrier,” as used herein, means a non-toxic, inert solid, semi-solid or liquid filler, diluent, encapsulating material or formulation auxiliary of any type. Exemplary pharmaceutically acceptable carriers may include a single ingredient or a combination of two or more ingredients. For example, to formulate the therapeutic composition as a topical composition, the pharmaceutically acceptable carrier may include a topical carrier. Suitable topical carriers may include, but are not limited to, one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof. Carriers for skin applications may include propylene glycol, dimethyl isosorbide, and water. For example, carriers for skin applications may include, but are not limited to, phosphate buffered saline, isotonic water, deionized water, monofunctional alcohols, symmetrical alcohols.

The pharmaceutically acceptable carrier of a topical composition may further include one or more ingredients selected from emollients, propellants, solvents, humectants, thickeners, powders, fragrances, pigments, and preservatives, all of which may be optional.

Suitable emollients may include, but are not limited to, stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin may include stearyl alcohol and polydimethylsiloxane. The amount of emollient(s) in a skin-based topical composition may be about 5% to about 95%.

Suitable propellants may include, but are not limited to, propane, butane, isobutane, dimethyl ether, carbon dioxide, nitrous oxide, and combinations thereof. The amount of propellant(s) in a topical composition may be about 0% to about 95%.

Suitable solvents may include, but are not limited to, water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents may include ethyl alcohol and homotopic alcohols. The amount of solvent(s) in a topical composition may be about 0% to about 95%.

Suitable humectants may include, but are not limited to, glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition may be about 0% to about 95%. The amount of thickener(s) in a topical composition may be about 0% to about 95%.

Suitable powders may include, but are not limited to, beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition may be about 0% to about 95%.

The amount of fragrance in a topical composition may be about 0% to about 0.5%, such as about 0.001% to about 0.1%.

Suitable pH adjusting additives may include, but are not limited to, HCl or NaOH in amounts sufficient to adjust the pH of a topical pharmaceutical composition.

Exemplary therapeutic compositions may have a pH of less than 8. In various instances, the therapeutic composition has a pH of 3.5 to 7.5; 4 to 7; 4.5 to 6.5; or 5 to 6. In various instances, the therapeutic composition may have a pH of no greater than 7.5; no greater than 7; no greater than 6.5; no greater than 6; no greater than 5.5; no greater than 5; no greater than 4.5; or no greater than 4. In various instances the therapeutic composition may have a pH of no less than 3.5; no less than 4; no less than 4.5; no less than 5; no less than 6.5; no less than 7; or no less than 7.5.

### E. Methods of Preparing Therapeutic Composition

The therapeutic compositions described herein may be prepared by various suitable methods. The particular method may depend on the specific therapeutic composition being prepared. In various instances, exemplary therapeutic compositions may be prepared by: (1) preparing a protein support solution; (2) mixing a rhamnolipid with the protein support solution; and (3) electrospinning the mixture of rhamnolipid and protein support solution. In some instances, one or more additional components (e.g., an additional therapeutic agent) may be mixed with the rhamnolipid and protein support solution, followed by electrospinning. In some instances, following electrospinning, the therapeutic composition may be desiccated to dryness.

Exemplary protein support solutions may generally comprise a protein and an organic solvent. Exemplary proteins include, without limitation, collagen, elastin, albumin, casein, fibrin, fibroin, keratin, tropoelastin, or a combination thereof. Exemplary organic solvents include, without limitation, a polyamide, a polyacrylonitrile, a polyacetal, a polyester, or a polyketone, or a combination thereof. In some instances, the organic solvent is ethanol (EtOH), ethyl formate, hexafluoro-2-propanol (HFIP), cyclic ethers, acetone, acetates of C2 to C5 alcohol, glyme or dimethoxyethane, methylethyl ketone, dipropyleneglycol methyl ether, lactones, 1,4-dioxane, 1,3-dioxolane, ethylene carbonate, dimethylcarbonate, diethylcarbonate, benzene, toluene, benzyl alcohol, p-xylene, N-methyl-2-pyrrolidone, dimethylformamide, chloroform, 1,2-dichloromethane (DCM), morpholine, dimethylsulfoxide (DMSO), hexafluoroacetone sesquihydrate (HFAS), anisole and mixtures thereof.

In various instances, mixing the rhamnolipid with the protein support solution may be performed at a temperature of 0° C. to 100° C.; 5° C. to 95° C.; 10° C. to 90° C.; 15° C. to 85° C.; 20° C. to 80° C.; 25° C. to 75° C.; 30° C. to 70° C.; 35° C. to 65° C.; 40° C. to 60° C.; or 45° C. to 55° C. In some instances, mixing the rhamnolipid with the protein support solution may be performed at a temperature of no greater than 100° C.; no greater than 90° C.; no greater than 80° C.; no greater than 70° C.; no greater than 60° C.; no greater than 50° C.; no greater than 40° C.; no greater than 30° C.; or no greater than 20° C. In some instances, mixing the rhamnolipid with the protein support solution may be performed at a temperature of no less than 0° C.; no less than 10° C.; no less than 20° C.; no less than 30° C.; no less than 40° C.; no less than 50° C.; no less than 60° C.; no less than 70° C.; or no less than 80° C.

In various instances, electrospinning may be performed at a temperature of 0° C. to 100° C.; 5° C. to 95° C.; 10° C. to 90° C.; 15° C. to 85° C.; 20° C. to 80° C.; 25° C. to 75° C.; 30° C. to 70° C.; 35° C. to 65° C.; 40° C. to 60° C.; or 45° C. to 55° C. In some instances, electrospinning may be performed at a temperature of no greater than 100° C.; no greater than 90° C.; no greater than 80° C.; no greater than 70° C.; no greater than 60° C.; no greater than 50° C.; no greater than 40° C.; no greater than 30° C.; or no greater than 20° C. In some instances, electrospinning may be performed at a temperature of no less than 0° C.; no less than 10° C.; no less than 20° C.; no less than 30° C.; no less than 40° C.; no less than 50° C.; no less than 60° C.; no less than 70° C.; or no less than 80° C.

In various instances, electrospinning may be performed at a relative humidity of 0% to 70%; 5% to 65%; 10% to 60%; 15% to 55%; 20% to 50%; 25% to 45%; or 30% to 40%. In some instances, electrospinning may be performed at a relative humidity of no greater than 70%; no greater than 60%; no greater than 50%; no greater than 40%; no greater than 30%; no greater than 20%; or no greater than 10%. In some instances, electrospinning may be performed at a relative humidity of no less than 0%; no less than 10%; no less than 20%; no less than 30%; no less than 40%; or no less than 50%.

In various instances, electrospinning may be performed at a voltage of 5 kV to 50 kV; 5 kV to 45 kV; 10 kV to 40 kV; 15 kV to 35 kV; or 20 kV to 30 kV. In some instances, electrospinning may be performed at a voltage of no greater than 50 kV; no greater than 45 kV; no greater than 40 kV; no greater than 35 kV; no greater than 30 kV; no greater than 25 kV; no greater than 20 kV; no greater than 15 kV; or no greater than 10 kV. In some instances, electrospinning may be performed at a voltage of no less than 5 kV; no less than 10 kV; no less than 15 kV; no less than 20 kV; no less than 25 kV; no less than 30 kV; no less than 35 kV; no less than 40 kV; or no less than 45 kV.

In various instances, electrospinning may be performed at a flow rate of 0.01 mL/hr to 5 mL/hr; 0.05 mL/hr to 4.5 mL/hr; 0.1 mL/hr to 4 mL/hr; 0.2 mL/hr to 3.5 mL/hr; 0.3 mL/hr to 3 mL/hr; 0.4 mL/hr to 2.5 mL/hr; 0.5 mL/hr to 2 mL/hr; 0.6 mL/hr to 1.9 mL/hr; 0.7 mL/hr to 1.8 mL/hr; 0.8 mL/hr to 1.7 mL/hr; 0.9 mL/hr to 1.6 mL/hr; 1.0 mL/hr to 1.5 mL/hr; 1.1 mL/hr to 1.4 mL/hr; or 1.2 mL/hr to 1.3 mL/hr. In various instances, electrospinning may be performed at a flow rate of no greater than 5 mL/hr; no greater than 4.5 mL/hr; no greater than 4 mL/hr; no greater than 3.5 mL/hr; no greater than 3 mL/hr; no greater than 2.5 mL/hr; no greater than 2 mL/hr; no greater than 1.5 mL/hr; no greater than 1.0 mL/hr; no greater than 0.9 mL/hr; no greater than 0.8 mL/hr; no greater than 0.7 mL/hr; no greater than 0.6 mL/hr; no greater than 0.5 mL/hr; no greater than 0.4 mL/hr; no greater than 0.3 mL/hr; no greater than 0.2 mL/hr; no greater than 0.1 mL/hr; or no greater than 0.05 mL/hr.

### III. Wound Care Dressings

In some instances, the therapeutic compositions may be configured for incorporation into a wound dressing material including, but not limited to, a bandage, a wipe, a sponge, a mesh, a dressing, a gauze, a patch, a pad, tape, or a wrap, or other wound dressing material. In various instances, the therapeutic composition may be used to saturate, impregnate, cover, coat, or otherwise be incorporated into a wound dressing material.

Exemplary wound dressings should be capable of attachment to the wound site, non-toxic, and elicit no more than a minimal allergenic response. Additionally, exemplary wound dressings should prevent bacteria or viral infections from entering the wound.

### IV. Methods of Wound Treatment

Exemplary therapeutic compositions may be used for wound treatment. Exemplary methods of treating a wound of a subject may comprise applying the therapeutic composition to a surface of the subject's wound. As used herein. a “wound” refers to a burn, an abrasion, a laceration, a lesion, an ulcer, or a sore. Wounds include diabetic foot ulcers, severe burns, tumor excision, or trauma. A wound includes damage to the skin, and can include, for example damage caused by trauma, burn, surgery, or other type of damage. In some instances, the method of treating a skin wound enhances wound healing, prevents pathogen biofilm formation, inhibits pathogen growth and proliferation, inhibits sepsis, and prophylactically prevents the formation of biofilm or growth of pathogens. In some instances, the wound is skin damage, a burn, an abrasion, a laceration, an incision, a sore, a puncture wound, a penetration wound, a gunshot wound, or a crushing injury.

In various instances, the wound may contain one or more metal ions. The metal ions may comprise any metal ions that negatively impact the metabolic process of wound healing such as, but not limited to, uranyl (UO₂) ions, europium (Eu) ions, neodymium (Nd) ions, terbium (Tb) ions, dysprosium (Dy) ions, lanthanide (La) ions, copper (Cu) ions, aluminum (Al) ions, lead (Pb) ions, yttrium (Y) ions, praseodymium (Pr) ions, lutetium (Lu) ions, Cd, In, zinc (Zn) ions, iron (Fe) ions, mercury (Hg) ions, calcium (Ca) ions, strontium (Sr) ions, cobalt (Co) ions, nickel (Ni) ions, barium (Ba) ions, manganese (Mn) ions, magnesium (Mg) ions, rubidium (Rb) ions, potassium (K) ions, and combinations thereof. In some instances, the wound may contain depleted uranium (DU).

Exemplary methods of wound treatment may comprise applying the therapeutic composition in an amount effective to accelerate wound healing, promote wound closure, or cause wound regression. The applying the therapeutic composition may reduce, attenuate, or prevent bacterial growth or infection in the wound. In some instances, the therapeutic composition does not prevent human or mammalian cells from proliferating. In various instances, the therapeutic composition may be applied to the wound at least once daily. in some instances, the therapeutic composition may be applied to the wound at least twice daily.

In some instances, the therapeutic composition may be used alone, for example, for direct application to a wound. Thus, for example, the composition may be formulated as an ointment, a cream, a spray, a spritz, a mist, a liquid, a gel, a lotion, or a solution. In some instances, the therapeutic composition may be topically applied to a wound. In other instances, the therapeutic composition may be administered subcutaneously.

### V. Experimental Examples

### A. Materials and Methods

Equipment and Software. Membrane integrity, cell metabolism, and DNA content were measured utilizing Gen5 software in tandem with the Biotek ® Instrument INC Synergy HT. Incubation temperature was set to 37° C.

Preparation of Depleted Uranium Solution. Approximately 0.005 g of uranyl nitrate (UN) crystals, purchased from American Scientific were weighed and solubilized in 40 mL of Dulbecco's modified eagle medium (DMEM) containing 10% fetal bovine serum (FBS) (DMEM complete) from Gibco® to create an initial working stock solution. Uranyl nitrate stock underwent 60 seconds of pipette mixing to confirm proper disintegration prior to vacuum sterilization using a 0.22 μm filter. Molecular weight was used to calculate the appropriate starting molarity before utilizing weight-volume concentrations to obtain the following experimental dilutions: 0.1 μM, 0.2 μM, 1.0 μM, 2.0 μM, 10 μM, 20 μM made from the sterile-filtered stock solution.

Cell Culture of Neonatal Human Dermal Fibroblasts (hDFn). Passage one neonatal human dermal fibroblast (hDFn) cells were purchased from Cell Applications, Inc. (P1, lot #1734). The hDFn cells were incubated at 37° C., 5% CO, and passaged using a T-75 tissue culture flask from Corning, completing media changes every 48 hours until 90% confluency was met. Cells were subcultured at a density of 10×10⁵ cells/mL, frozen in DMEM containing 10% DMSO, and stored at −80° C. for 24 hours before being placed in liquid nitrogen vapor phase for long-term preservation. The subsequent process was repeated until passage 4 isolates were frozen down.

Rhamnolipid Solution. About 0.0125 g of rhamnolipids (Rha-C10-C10) from Glycosurf were weighed and diluted in 50 mL of 1× phosphate-buffered saline (PBS) to obtain a 200 μM rhamnolipid stock solution. The solution was heated prior to being vortexed for maximum dissolution and sterilized through a vacuum using a 0.22 μm filter. The molarity of rhamnolipid stock solution was found using actual mass (250 mg) and molecular weight to calculate experiment dilutions of 6.25 μM, 50 μM, and 100 μM.

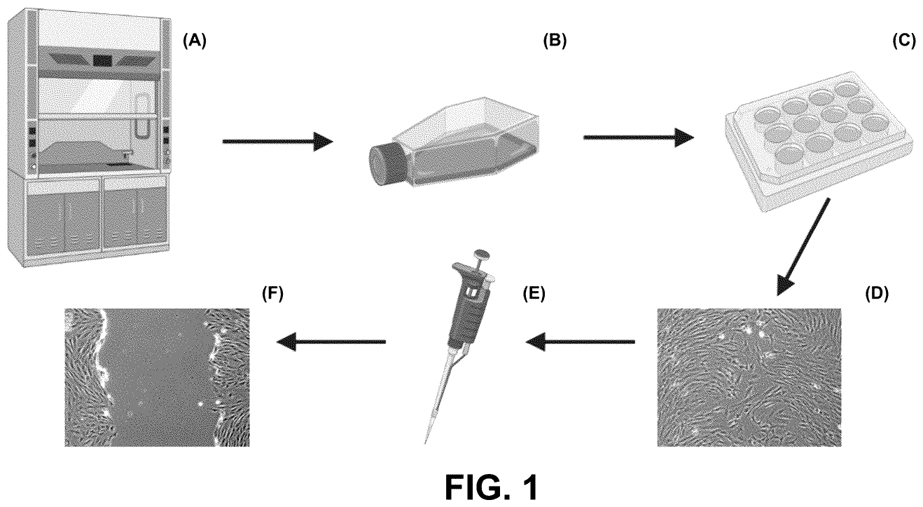

Cellular Migration Assay. Human neonatal dermal fibroblasts (hDFn) passage 4, were seeded into a T-75 flask at 10×10⁵ cells/mL density and grown in a standing incubator at 37° C., 5% CO₂. Once 90% confluence was reached, hDFn were subcultured into 12-well plates, and respective UN treatments with or without rhamnolipids were added. After 24 hours, contents were removed, and the monolayer of fibroblasts was scratched vertically using a sterile pipet tip creating the mock wound (FIG. 1). Wells were then washed twice with Hanks' Balanced Salt Solution (HBSS). Respective treatments were added back into wells and cellular migration rates were monitored and imaged over time (0 hours, 12 hours, and 24 hours). Percent closure rates were collected using MATLAB®.

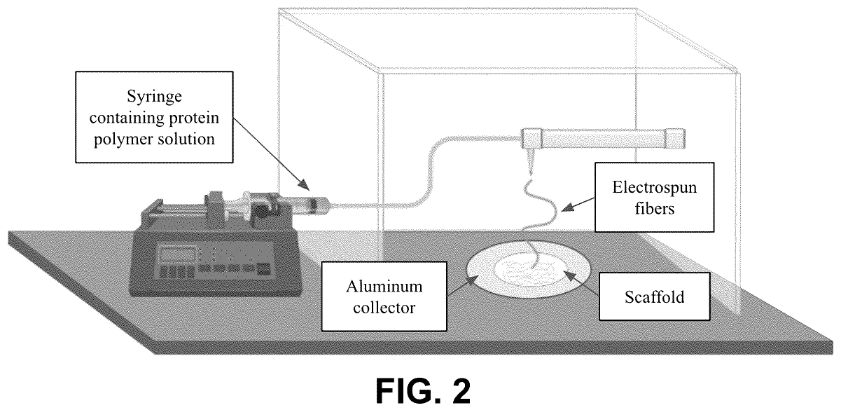

Construction of Electrospun Scaffolds. A protein polymer solution was prepared using a 10% bovine gelatin and hexafluoroisopropanol (HFIP) solvent mixture in addition to varying concentrations of rhamnolipids (1 wt %, 2.5 wt %, 5 wt %, and 10 wt %). The protein polymer solution was charged at a high voltage (20 kV) and a flow rate of 1 mL/hour with a 12 cm distance from the aluminum collector where fibers accumulated (FIG. 2). Electrospun scaffolds were left to sit in a desiccator overnight prior to crosslinking for 1.5 hours with 50% glutaraldehyde vapors and placed overnight uncovered in the desiccator once again. Scaffolds were placed 4 inches beneath UV rays for sterilization for 1 hour on each side.

Cellularization of Electrospun Scaffolds. Scaffold cellularization steps were completed aseptically inside the BioSafety cabinet in accordance with the laboratory's mammalian cell culture standard operating procedure. Biopsy punches of 10 mm segments were removed from the control and treatment scaffolds (1 wt %, 2.5 wt %, 5 wt %, and 10 wt % rhamnolipid) and placed inside each well of a 48-well plate using sterile forceps that were treated in 70% ethanol for a minimum of 5 minutes. Approximately 1.5 mL of 1× PBS was used to rinse each membrane and then aspirated. Scaffolds sat in the biosafety cabinet for 1 hour to dry and adhere to the bottom of each well. Cells were then suspended onto scaffolds at a density of 11,000 cells/mL and allowed to rest for 30 minutes before placing into the standing incubator (37° C., 5% CO₂) for 48 hours.

Scanning Electron Microscopy. Scanning electron microscopy (SEM) was used to assess changes in nanofiber structure and cellular biocompatibility in impregnated scaffolds at various magnifications. Following cellularization, scaffolds were rinsed with 1× PBS and fixed with 2.5% glutaraldehyde (GA) in Na-cacodylate for 1 hour at room temperature. GA was aspirated and rinsed with approximately 0.5 mL of Na-cacodylate thrice for 15 minutes each. Thereafter, 0.5 mL of 2% Osmium in DI water was placed in wells and allowed to sit at room temperature for 1 hour and then rinsed three times for 5 minutes each with DI water only. HDFn cells were dehydrated with approximately 0.5 mL of the following ethanol (EtOH) dilutions: 30%, 50%, 70%, and 95% EtOH for 10 minutes, and 100% EtOH for 10 minutes three times. While leaving 100% EtOH in wells, 0.5 mL of hexamethyldisilazane (HMDS) was added (50:50 mixture) for 5 minutes. Contents were removed and 100% HMDS was placed in wells and left for 5 minutes twice. Samples were left in the fume hood to air dry for 1 hour. After mounting samples onto aluminum stubs and coating with gold palladium for 10 seconds, samples were viewed and imaged at various magnifications using the SEM, assessing for X, Y, and Z.

PrestoBlue™ and CyQUANT™ Direct Assay. PrestoBlue™ and CyQuant™ (PBCQ) direct confirmation assay (from ThermoFisher®) for cell viability was used to assess the biocompatibility of hDFn cells in DMEM complete media with varying concentrations of rhamnolipids. Passage 5 hDFn cells were seeded at 11,200 cells/mL density into black wall, clear bottom 96-well tissue culture plates. The cells were placed inside the standing incubator at 37° C. and 5% CO₂ until reaching 70-90% confluence. Media was removed and replaced with the following rhamnolipid concentrations with an n=6 for 24 hours: 6.25 μM, 50 μM and 100 μM. The plate layout included blank wells containing DMEM complete media and the corresponding rhamnolipid concentrations. Following 24 hours, 100 μL was removed from each well and replaced with 10 μL of the PrestoBlue reagent. The plates were then incubated for 20 minutes at 37° C. and 5% CO₂ prior to being placed in the Biotek® Synergy HT fluorescence microplate reader at 590 nm with a gain value of 35. Afterward, 100 μL of the CyQuant reagent composition was added to all wells and incubated for 60 minutes. The plates were read using the same microplate reader at 528 nm with a gain of 70. The relative fluorescence units (RFU) were produced for all wells, correlating with the metabolic output per unit of DNA.

Statistical Analysis. Statistically significant differences were determined using GraphPad Prism. The results of the cellular migration assay were analyzed with a one-way ANOVA and Tukey post hoc evaluation. The PBCQ assay was analyzed using a one-way ANOVA and Tukey post hoc evaluation. For all experiments, a p-value<0.05 was considered statistically significant in this study.

Rhamnolipid vs. Thioglycolipid Comparative Scratch Assays. Human dermal neonatal fibroblasts (hDFn) were seeded in 3×12-well plates at 10,000 cell/cm². Media (Dulbecco's Modified Eagle Medium (DMEM)) was changed every 48 hours. Plates were imaged every 4 hours for 24 hours. Plates were analyzed via MATLAB® and RStudio®.

### B. Results

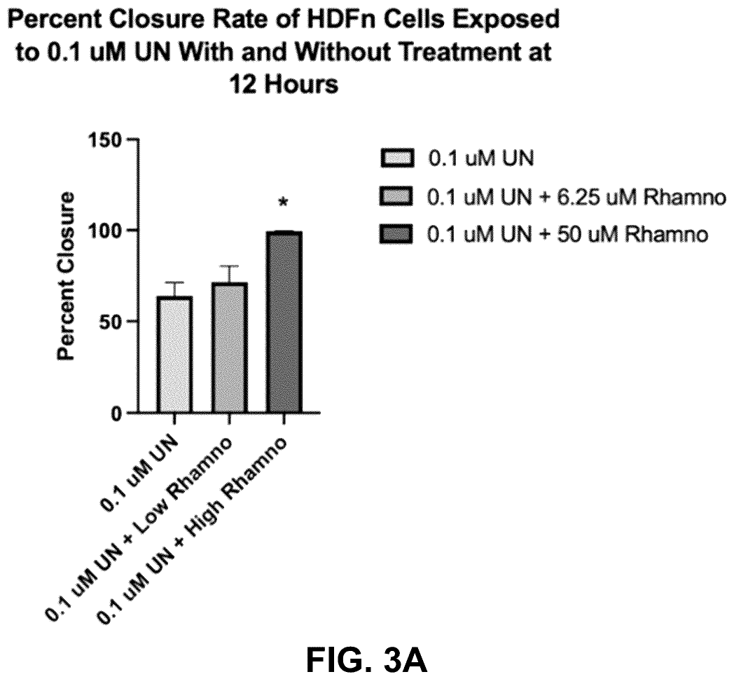

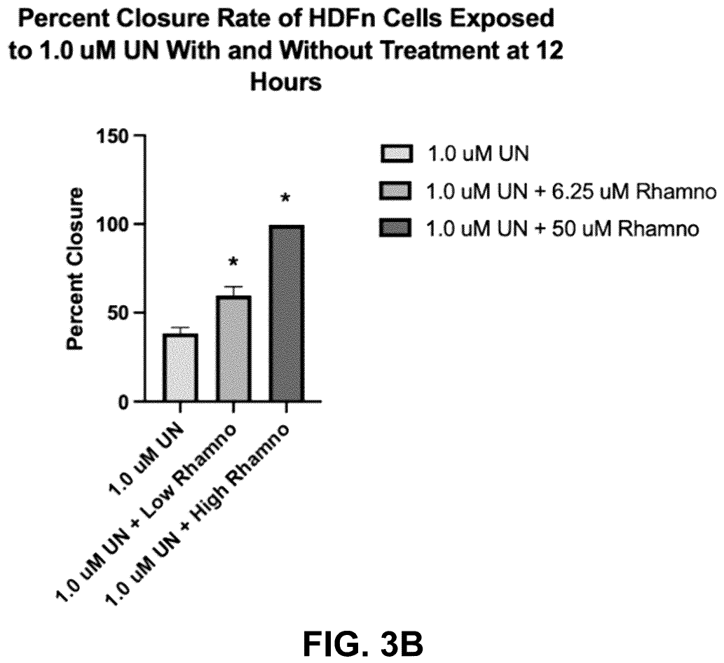

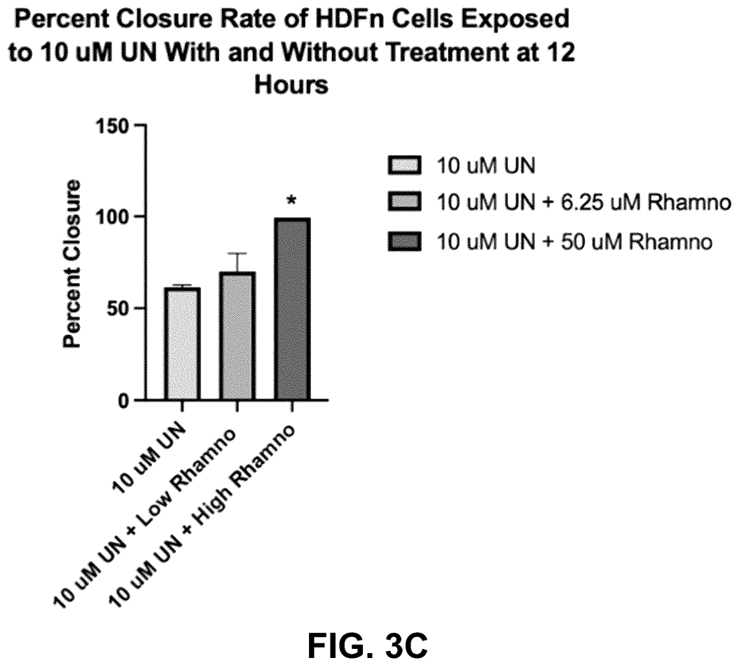

Cellular Migration Assay. The cellular migration assay was used to determine if using rhamnolipids as a treatment for depleted-uranium toxicity would result in favorable hDFn cell migration, meaning the closure rate was not impaired or delayed in comparison to the control. The cellular migration assay results suggest that the migration rate, measured by percent closure, is increased in the presence of rhamnolipids, despite exposure to DU (Tukey's, p<0.05) (FIGS. 3A-3C). HDFn cells exposed to 0.1 μM, 1.0 μM and 10 μM UN resulted in a statistically significant increase in percent closure as early as 12 hours post-wound with the high concentration of rhamnolipids (50 μM) in comparison to the control (0.1 μM+50 μM: p-value=0.021; 1.0 μM+50 μM: p-value<0.001; 10 μM+50 μM: p-value=0.009). The cells exposed to 1.0 μM UN displayed a statistically significant increase in percent closure 12 hours post-wound with the low concentrations of rhamnolipids (6.25 μM) (1.0 μM+6.25 p-value=0.012). Throughout the experiment, 24 hours post-wound was considered closed as there was no statistically significant difference in mean percent closure across the different treatments.

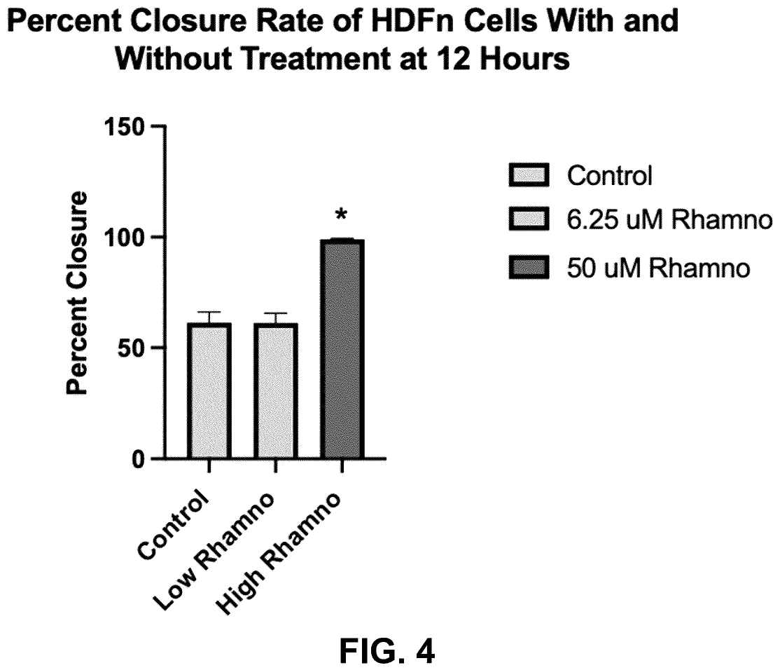

Fibroblast cell migration was evaluated under low and high rhamnolipid concentration conditions, in the absence of UN (F=32.81, p<0.001) (FIG. 4). There was a statistically significant increase in cellular migration in wells treated with a high dose of rhamnolipids (Tukey's, p=0.001). Furthermore, there was no statistically significant difference in mean percent closure between the control and the low concentration of rhamnolipids.

Scanning Electron Microscopy. SEM images were taken to visually determine if the hDFn cells were able to demonstrate normal cellular behavior (elongation of filipodia, ability to adhere onto structure) on scaffolds impregnated with rhamnolipids compared to the control with no rhamnolipids. The bare control and treatment scaffolds, containing no cells, were mounted onto aluminum stubs, and coated with gold palladium for 10 seconds. Cellularized scaffolds (both control and treatment) were seeded with hDFn cells and fixed using glutaraldehyde, dehydrated with hexamethyldisilane, air-dried for 1 hour, sputter coated with gold palladium, and mounted with carbon tape. On the SEM, the SE2 and InLens detectors were used, as well as an accelerating voltage of 2.00 kV-5.00 kV with magnifications ranging from 300× and 999×.

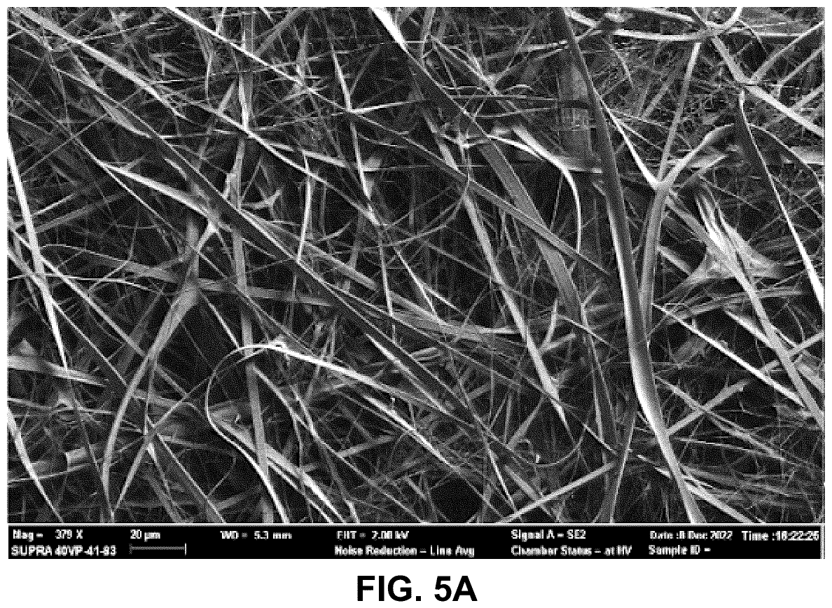

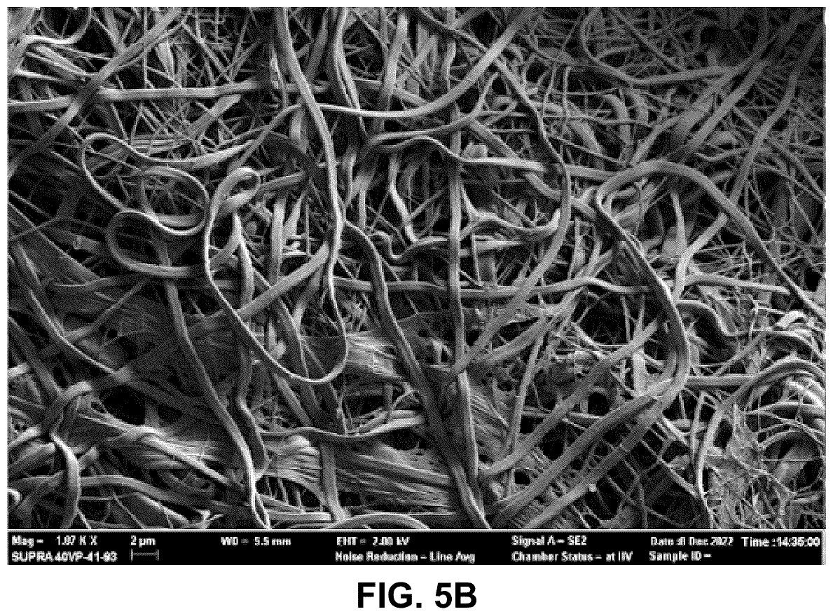

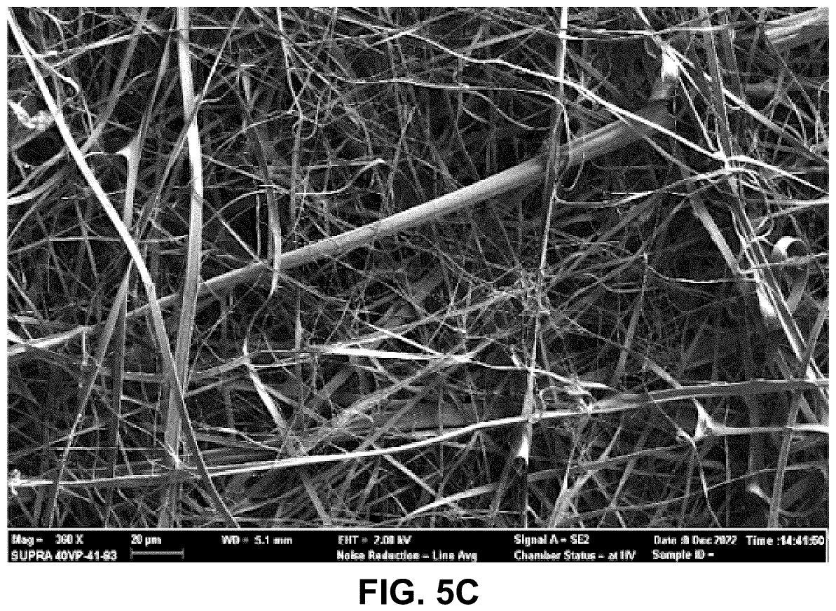

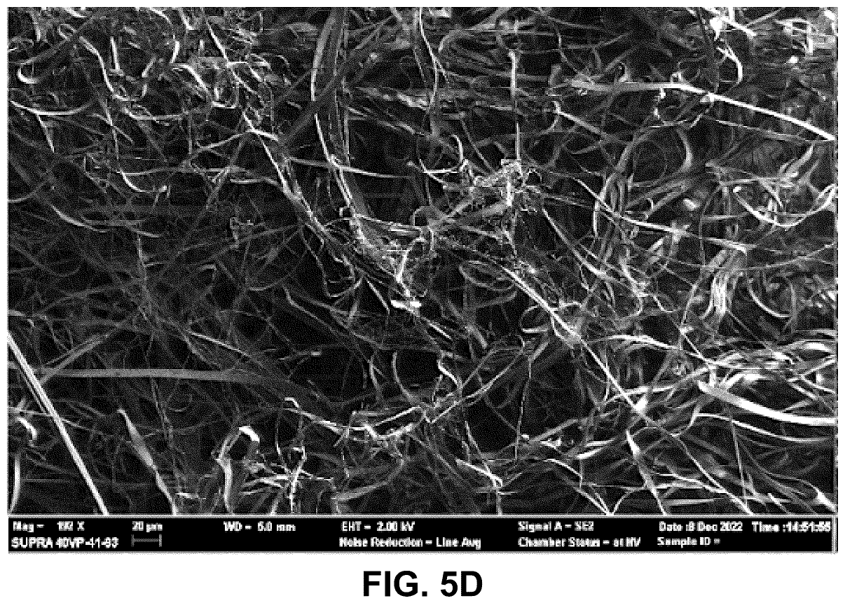

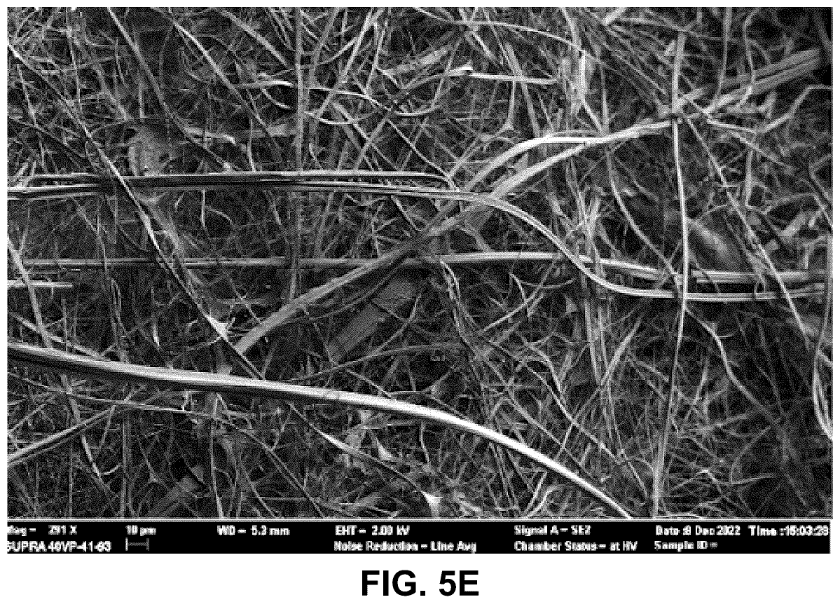

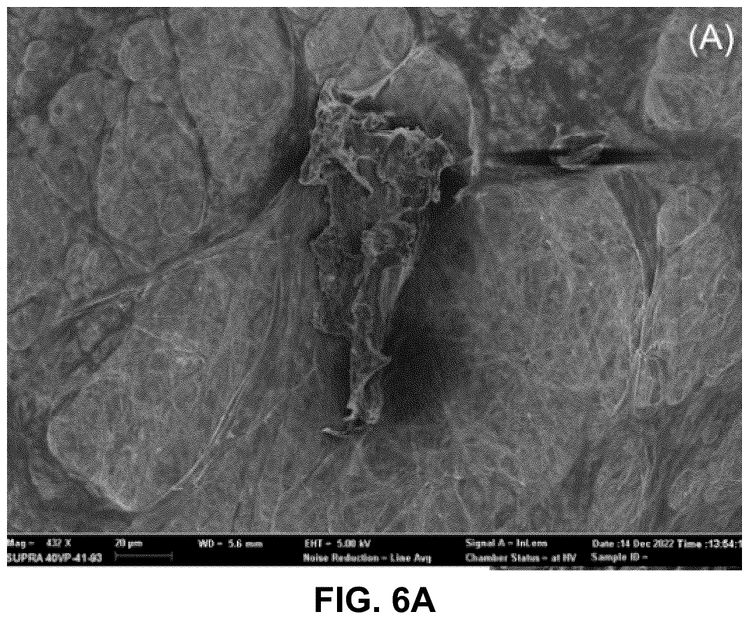

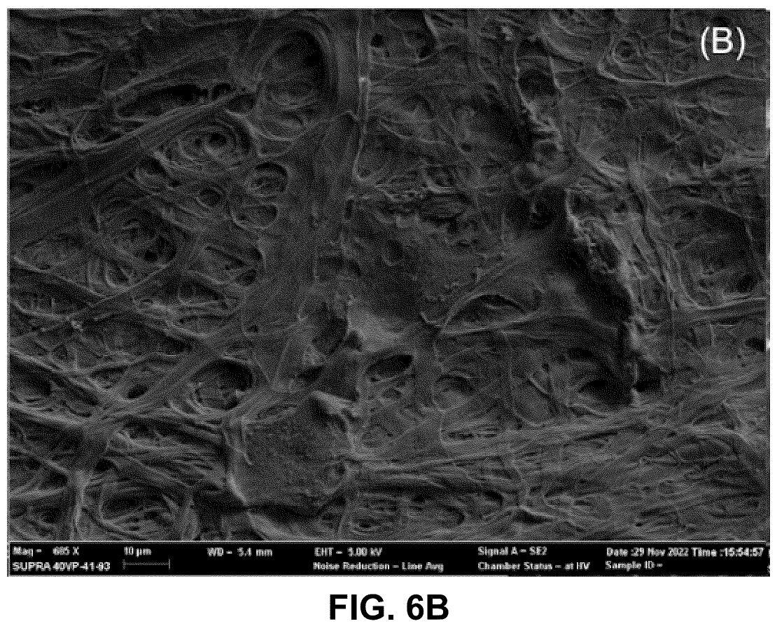

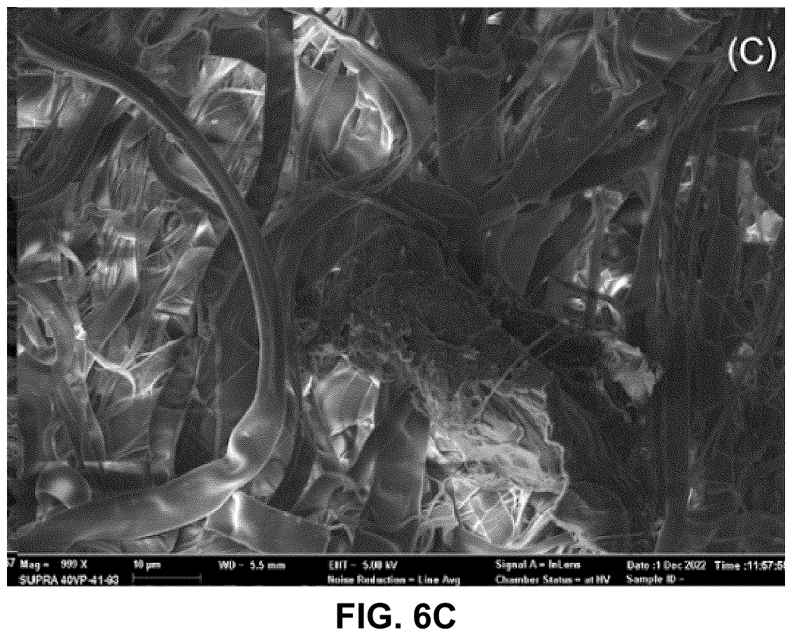

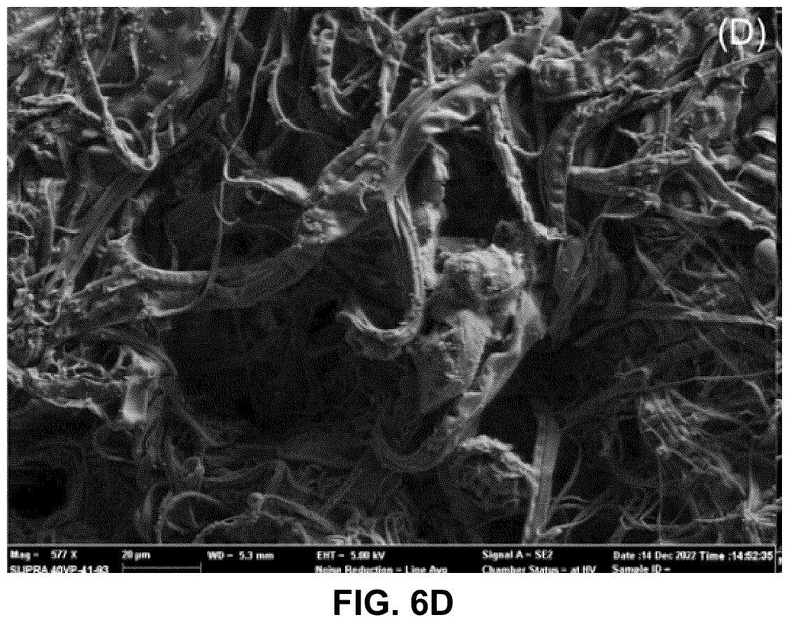

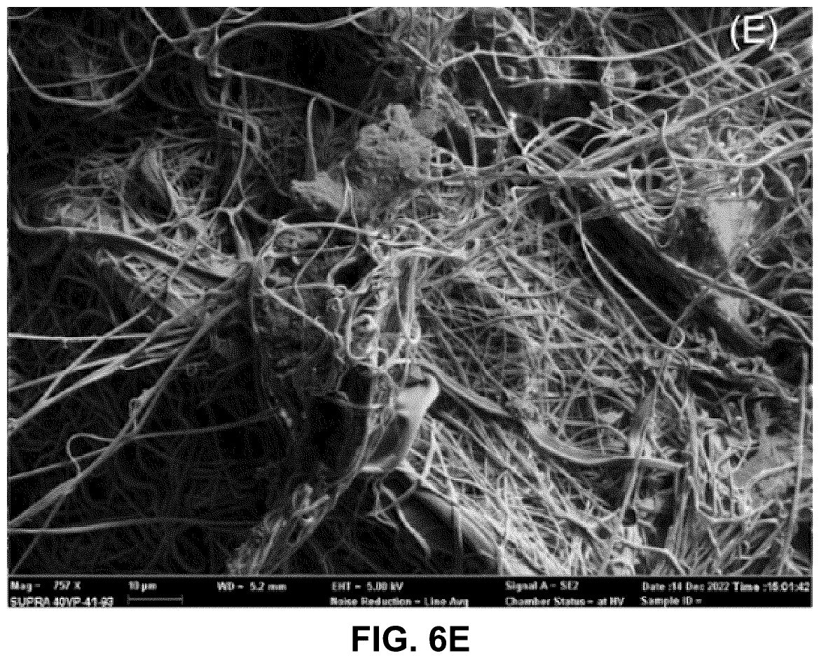

The control scaffold, the SEM image of which is shown in FIG. 5A, was not impregnated with rhamnolipids, or had hDFn cells seeded onto it. In particular, the observable porosity is not structurally different in FIG. 5A compared to the other images (FIGS. 5B-5E) depicting impregnated scaffolds without cells. As the percentage of rhamnolipids increased, the porosity of the scaffold remained relatively similar to the control. Furthermore, hDFn cells grew and migrated onto all the treatment scaffolds (FIGS. 6A-6E) at 1 wt %, 2.5 wt %, 5 wt %, and 10 wt %. Visually, the hDFn cells appeared to have been able to adhere and colonize onto impregnated scaffolds which could be further explored to better understand the cytocompatibility of rhamnolipids and if their morphology is influenced by the concentration of the surfactant. PrestoBlue™ and CyQUANT™ Direct Assay. The PrestoBlue™ and CyQUANT™ Direct assays were used to determine the cellular compatibility of hDFn cells when in the presence of rhamnolipids. The PrestoBlue™ assay is a resazurin-based solution when upon entering a living cell, the reagent is reduced to resorufin which is red in color and fluorescent. The overall health of the cell can be observed by changes in fluorescence and subsequently absorbance. Metabolically active cells constantly convert the PrestoBlue™ reagent whereas non-viable cells cannot reduce the dye and do not produce a change in signal. The CyQUANT™ Direct assay is a cell-permeant green nucleic acid stain that is combined with a masking dye reagent. The masking dye stops the staining of non-viable cells or cells with compromised cell membranes, resulting in only the viable cells being stained. The CyQUANT™ Direct assay measures proliferation as well as membrane integrity. The data was evaluated using GraphPad Prism to run a one-way ANOVA.

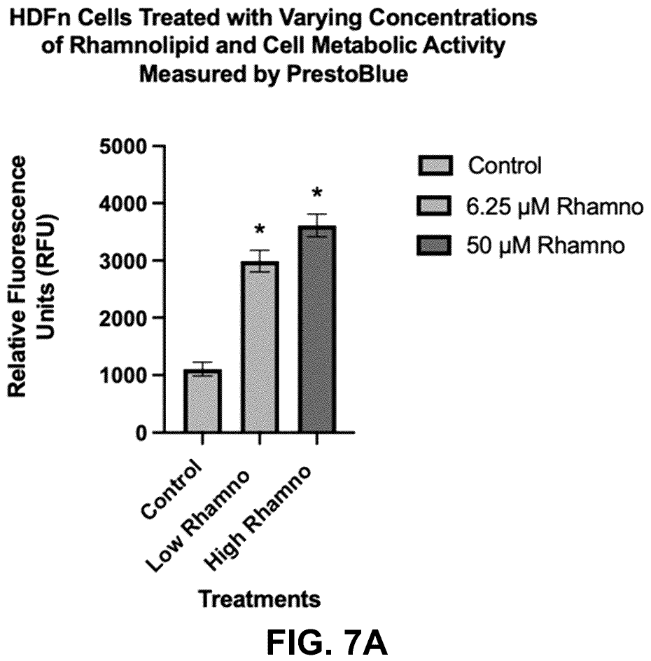

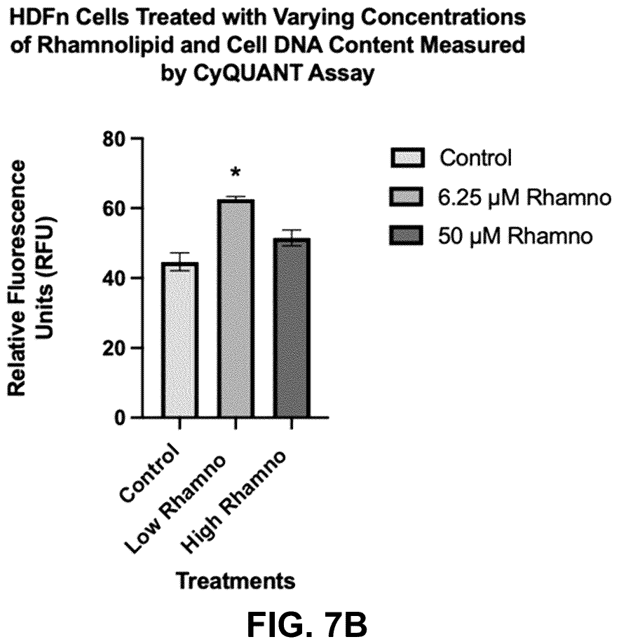

The results of the PrestoBlue™ (FIG. 7A) assay demonstrated a statistically significant increase in metabolic activity from percent concentrations of 6.25 μM (Tukey's, p<0.001), and 50 μM (Tukey's, p<0.001) compared to the control (FIGS. 7A-7B). A one-way ANOVA of the PrestoBlue™ assay resulted in a P-value<0.001 and an F-value of 57.16. The CyQUANT™ Direct assay (right graph) results demonstrated a statistically significant increase in cellular DNA content with the low concentration of rhamnolipids (Tukey's, p<0.001). A one-way ANOVA of the CyQUANT™ Direct assay resulted in a P-value<0.001 and an F-value of 20.00. Meanwhile, there was no statistically significant difference between the living DNA content of the high concentration of rhamnolipids compared to the control.

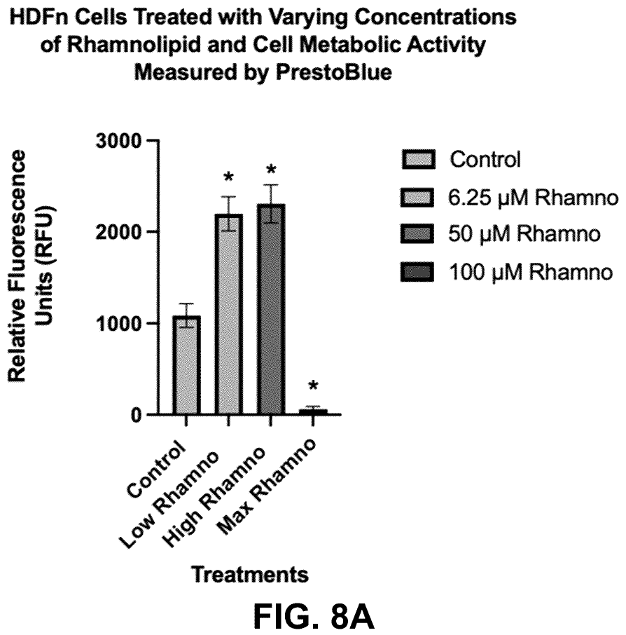

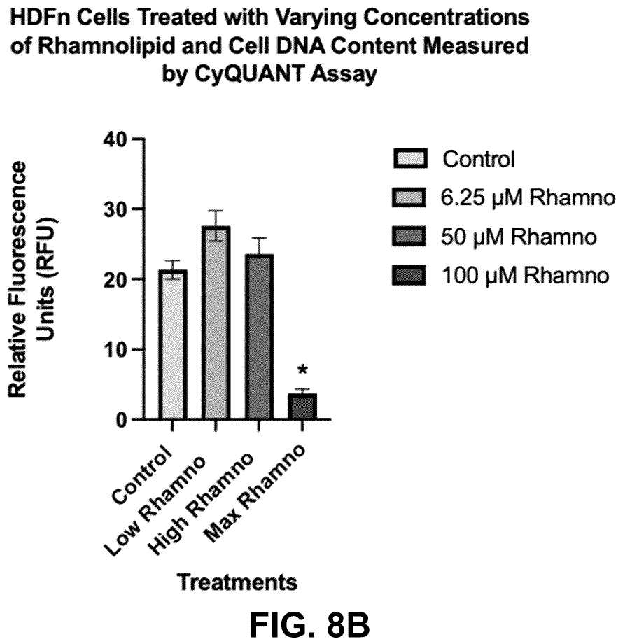

The results of the PrestoBlue™ (FIG. 7A) assay suggested a statistically significant increase in metabolic activity from percent concentrations of 6.25 μM (Tukey's, p<0.001), and 50 μM (Tukey's, p<0.001) compared to the control (FIGS. 7A-7B). A one-way ANOVA of the PrestoBlue™ resulted in a P-value<0.001 and an F-value of 45.85. Meanwhile, there was a statistically significant decrease in metabolic activity with the maximum concentration of rhamnolipids (100 μM) in comparison to the control (FIGS. 8A-8B). The CyQUANT™ Direct assay (right graph) demonstrated a statistically significant decrease in cellular DNA content with the 100 μM concentration of rhamnolipids as compared to the control. A one-way ANOVA of the CyQUANT™ Direct assay resulted in a P-value<0.001 and an F-value of 37.04. Furthermore, there was no statistically significant difference between the living DNA content of the low and high concentrations of rhamnolipids in comparison to the control. The max concentration of rhamnolipids could provide a threshold for usable dosages of the treatment in future studies.

The current study focused on leveraging rhamnolipid's metal-binding ability to sequester wounds contaminated with depleted uranium. It was hypothesized and confirmed that utilizing rhamnolipids as a treatment for depleted uranium toxicity would result in favorable hDFn cell migration.

FIGS. 3A-3C graphically show percent closure rates of hDFn cells exposed to depleted uranium (DU) with and without treatment. As shown in FIGS. 3A-3C, the results of the assay at 12 hours demonstrated a statistically significant increase in the percent closure rate (p-value<0.05) of cells with the high concentration of rhamnolipids (50 μM) regardless of the presence of DU at all concentrations (0.1 μM, 1.0 μM, 10 μM. This data shows that rhamnolipids neutralized the harmful effects of DU and allow cells to close at a higher rate despite exposure to heavy metals. Similarly, in FIG. 4, there is a trend shown with cells and the 50 μM of rhamnolipids. After 12 hours, the hDFn cells were observed to have increased in percent closure (p-value<0.05), suggesting that outside of acting as a chelator, rhamnolipids surfactant abilities were able to excite the hDFn cells' ability to migrate toward each other.

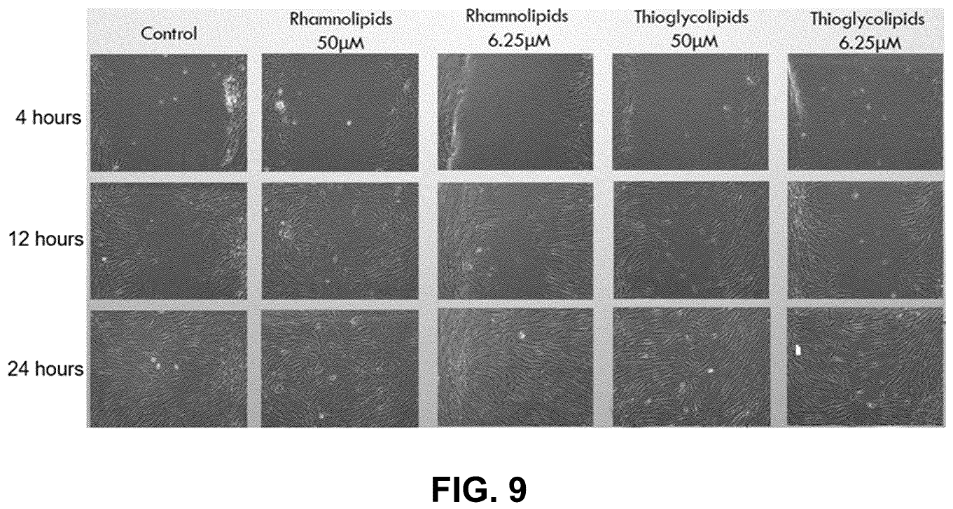

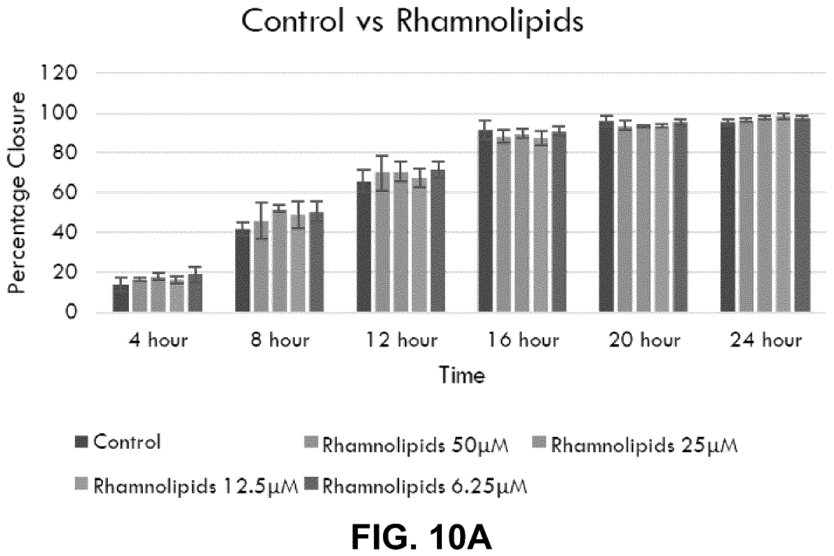

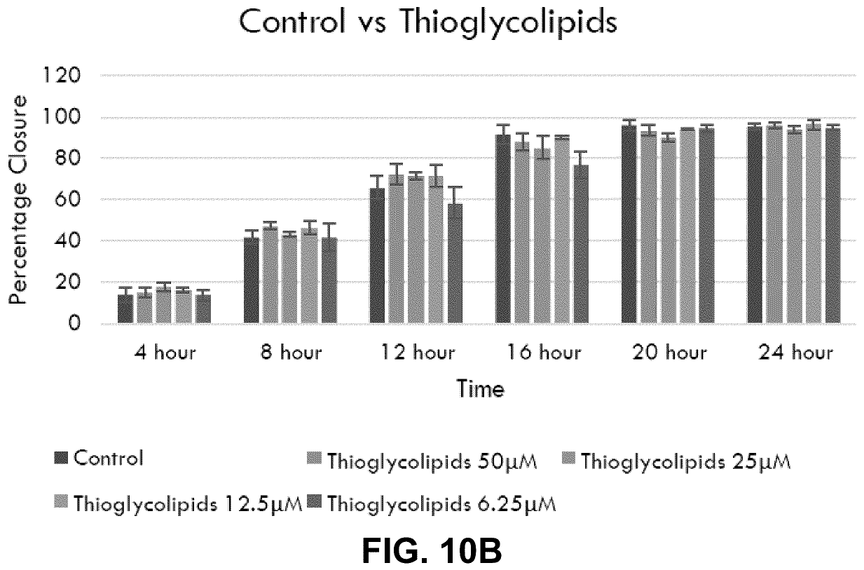

Rhamnolipid vs. Thioglycolipid Comparative Scratch Assays. SEM images of scratch assay results comparing the wound healing capabilities of rhamnolipid compositions versus thioglycolipid compositions over a 24-hour time period indicate that rhamnolipid compositions provide faster healing than thioglycolipid compositions (FIG. 9), particularly as observed at the 4-hour and 8-hour time marks (FIGS. 10A-10B).

### C. Discussion

This study is among the first to describe the effects of rhamnolipids as a treatment against acute in vitro DU exposure to hDFn cells in wound healing. However, other methods have described increased wound healing in vivo by applying di-rhamnolipids onto burn wounds on Swiss—Webster mice to measure if there was an increase in wound healing. The results from the study found that the wound closure rate measured on days 14, 21, and 28 was faster in mice that were administered the 0.1 wt % di-rhamnolipid ointment than in those that were not administered the rhamnolipid ointment. Moreover, these findings suggest that those who experience a delay in wound healing following exposure to DU may utilize rhamnolipids in assisting fibroblast migration and helping improve wound healing.

In addition, this research investigated whether these novel wound healing devices were able to be incorporated with unique chemical agents to facilitate delivery into the wound bed to de-contaminate the tissue from depleted uranium exposure. As shown in the SEM images of FIGS. 5A-5E, scaffolds were able to undergo the high voltage of the electrospinner with all concentrations of the treatment (1 wt %, 2.5 wt %, 5 wt %, and 10 wt %). The resulting SEM images supported this prediction and gave a sufficient indication of the visual presence of rhamnolipids in the scaffolds as there is a slight variation in thickness as percentages increase, with no overall visual change in structure. Subsequently, it was hypothesized that hDFn cells would continue to exhibit normal cellular behavior and the ability to colonize those electrospun scaffolds impregnated with rhamnolipids. The visual behavior of the cells showed indications of normal cell behavior, as fibroblasts are able to elongate along the scaffold structure at all concentrations of the treatment. As shown in the SEM images of FIGS. 6A-6E, hDFn cells colonized and adhered to impregnated scaffolds, indicating signs of a favorable environment.

The biocompatibility of rhamnolipids and hDFn cells was also assessed. Acute 24-hour exposure of skin cells to various concentrations of rhamnolipids was evaluated. It was hypothesized that when hDFn cells were cultured in the presence of the biosurfactant, rhamnolipid, hDFn cells would continue to exhibit favorable cellular processes. This hypothesis was supported by the PrestoBlue™ and CyQUANT™ Direct assays to determine the cellular compatibility of hDFn cells when in the presence of rhamnolipids. The PrestoBlue™ assay is a resazurin-based solution where the reagent is red in color and fluorescent. The overall metabolic activity of the cell can be observed by changes in fluorescence and absorbance. The CyQUANT™ Direct assay measures living DNA content and is a cell-permeant green nucleic acid stain that is combined with a masking dye reagent that hinders the staining of non-viable cells or cells with compromised cell membranes, resulting in only the viable cells being stained. As shown in FIGS. 7A-7B, a one-way ANOVA test was used to statistically analyze the results of the PBCQ data followed by Tukey's post hoc test.

The one-way ANOVA for the PrestoBlue™ produced a p-value<0.001, meaning the null is rejected in favor of the alternative indicating that there is a difference in means. Moreover, the post hoc Tukey's test supports this claim as both the low and high concentrations were found to have a significant increase in metabolic activity in comparison to the control with a p-value<0.001 for each. There is a similar trend shown in FIGS. 8A-8B, where again the one-way ANOVA of the PrestoBlue™ resulted in a P-value<0.001 and the low and high concentrations were found to have a significant increase in metabolic activity in comparison to the control with a p-value<0.001.

The importance of these findings indicates rhamnolipids outside of their ability to bind to heavy metals, are able to stimulate an increase in hDFn cell activity. Since hDFn cells are vital in the proliferation stage of wound healing, future studies could investigate rhamnolipids to aid in accelerating the wound healing process. However, the max concentration of the treatment (100 μM) resulted in a significant decrease in metabolic activity with a p-value <0.001. Decreased metabolism with the max concentration is significant for a couple of reasons. First, these results help define a threshold of usable dosages for rhamnolipids in future studies.

The one-way ANOVA for the CyQUAT™ direct assay showed a p-value<0.001, which means the null hypothesis was rejected in favor of the alternative. Tukey's post hoc test supports this claim as the low concentration of rhamnolipids was found to have a significant increase in DNA content in comparison to the control with a p-value<0.001. Indicating that the rhamnolipids promoted an increase in living cells in its presence. In addition, FIGS. 8A-8B showed the one-way ANOVA of the CyQUAT™ direct assay resulted in a P-value<0.001. However, there was no significant difference between the living DNA content of the low and high concentrations of rhamnolipids in comparison to the control as shown with Tukey's test. The importance of no significant increase or decrease in DNA content between the low and high treatments to the control indicates that there were no comparable differences in living DNA in cells with and without treatment present. Furthermore, the max concentration of rhamnolipids demonstrated a significant decrease in living DNA content with a p-value<0.001. A decrease in DNA content of hDFn cells with the max concentration is notable as the data helps define a firm threshold of usable concentrations of rhamnolipids in future studies.

In summary, the current study demonstrated that treating DU-exposed hDFn cells with rhamnolipids by utilizing the established cellular migration assay, demonstrated a surge in cellular migration and proliferation despite the presence of the heavy metal. SEM images illustrated the cytocompatibility between rhamnolipid-impregnated scaffolds and hDFn cells through their ability to grow and migrate into the structure at all concentrations of the treatment. The cellular biocompatibility of rhamnolipids was measured using the PBCQ assay where low and high concentrations of the treatment led to an increase in cell metabolism and showed no significant decrease in viability aside from the max dosage which may serve as a threshold for future studies. These findings are among the first to explore the effects of rhamnolipids as a treatment against internal DU exposure by using electrospun scaffolds alongside hDFn cells during the proliferation stage of wound healing to provide a healing therapeutic to treat those individuals affected by chronic exposure to U-contaminated water throughout the American Southwest.

The breadth and scope of the present disclosure should not be limited by any of the above-described exemplary aspects but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A therapeutic composition comprising:
a rhamnolipid, and
an electrospun support comprising a biomaterial;
wherein the rhamnolipid is impregnated within the electrospun support.

2. The therapeutic composition according to claim 1, the electrospun support comprising a naturally occurring biomaterial.

3. The therapeutic composition according to claim 2, the naturally occurring biomaterial comprising collagen, elastin, albumin, casein, fibrin, fibroin, keratin, tropoelastin, or a combination thereof.

4. The therapeutic composition according to claim 1, the electrospun support comprising a synthetic biomaterial.

5. The therapeutic composition according to claim 4, the synthetic biomaterial comprising polylactide (PLA), polyglycolide (PGA), poly(lactide-co-glycolide) (PLGA), polycaprolactone (PCL), polyvinylidene fluoride (PVDF), polydioxanone trimethylene carbonate (PTMC), poly(Bisphenol A carbonate) (PC), polycyanoacrylate, a polyhybroxyalkonate (PHA), a poly(ortho ester) (POE), or a combination thereof.

6. The therapeutic composition according to claim 1, the rhamnolipid comprising a mono-rhamnolipid, a di-rhamnolipid, or a combination thereof.

7. The therapeutic composition according to claim 6, the rhamnolipid comprising a rhamnolipid of formula (I): 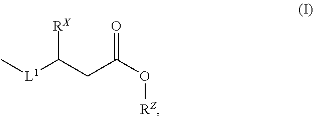
wherein:
R^{X} 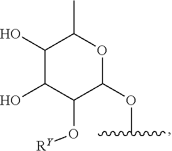
where R^{Y} is hydrogen or 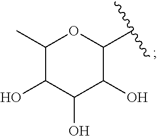 [SMILES: *C1OC(C)C(O)C(O)C1O]
R^{Z} is hydrogen or 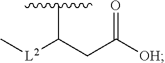
L¹ is C₂₋₁₈alkylene or C₂₋₁₈alkenylene; and
L² is C₂₋₁₈alkylene or C₂₋₁₈alkenylene.

8. The therapeutic composition of claim 7, wherein R^{X} is

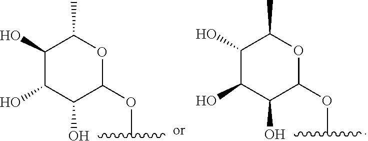 [SMILES: *OC1O[C@@H](C)[C@H](O)[C@@H](O)[C@H]1O ; *OC1O[C@H](C)[C@@H](O)[C@H](O)[C@@H]1O]

9. The therapeutic composition of claim 7, wherein R^{X} is

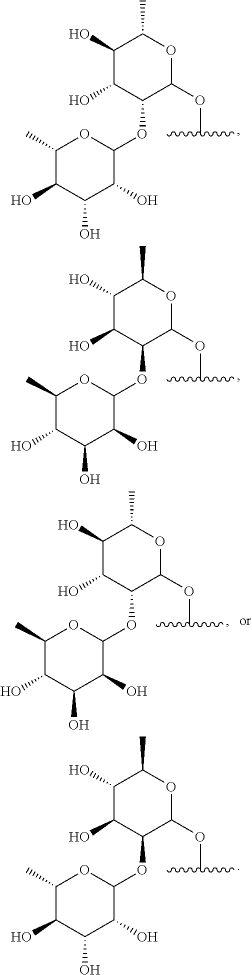

10. The therapeutic composition of claim 5, wherein the rhamnolipid of formula (I) is a rhamnolipid of formula (I-a), (I-b), (I-aa), (I-bb), (I-ab), or (I-ba):

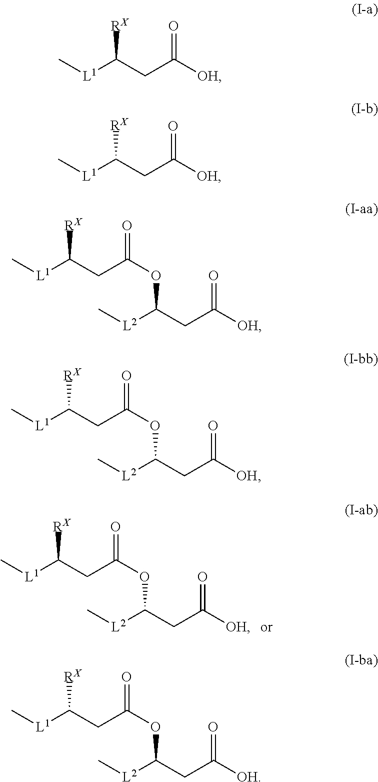

11. The therapeutic composition of claim 10, wherein L¹ is C₂₋₁₄alkylene or C₂₋₁₄alkenylene.

12. The therapeutic composition of claim 10, wherein L² is C₂₋₁₄alkylene or C₂₋₁₄alkenylene.

13. The therapeutic composition according to claim 7, where the rhamnolipid of formula (I) has enantiomeric purity in excess of 90.0%.

14. The therapeutic composition according to claim 1, wherein the rhamnolipid comprises a synthetically prepared rhamnolipid.

15. The therapeutic composition according to claim 1, wherein the rhamnolipid comprises a naturally occurring rhamnolipid.

16. The therapeutic composition according to claim 1, wherein the rhamnolipid is present in the therapeutic composition at a concentration of greater than 1 μM.

17. The therapeutic composition according to claim 16, wherein the rhamnolipid is present in the therapeutic composition at a concentration of less than 10,000 μM.

18. The therapeutic composition according to claim 16, wherein the rhamnolipid is present in the therapeutic composition at a concentration of 3 μM to 100 μM.

19. The therapeutic composition according to claim 1, wherein the therapeutic composition has a pH of less than 8.

20. The therapeutic composition according to claim 19, wherein the therapeutic composition has a pH of 4.5-6.5.

21. The therapeutic composition according to claim 1, wherein the therapeutic composition further comprises an antibiotic, a steroid, an analgesic, a cannabinoid, a growth factor, or a combination thereof.

22. A wound care dressing comprising:
the therapeutic composition according to claim 1, and
a wound dressing material.

23. The wound care dressing of claim 22, wherein the wound dressing material is a bandage, a wipe, a sponge, a mesh, a gauze, a patch, a pad, a tape, or a wrap.

24. A method of treating a wound of a subject, the method comprising applying the therapeutic composition of claim 1 to a surface of the subject's wound.

25. A method of treating a wound of a subject, the method comprising applying the therapeutic the wound care dressing of claim 22 to a surface of the subject's wound.

26. The method according to claim 24, wherein the wound contains one or more metal ions.

27. The method according to claim 26, wherein the wound contains depleted uranium.

28. The method according to claim 25, wherein the wound contains one or more metal ions.

29. The method according to claim 28, wherein the wound contains depleted uranium.
